(19) United States
(12) Patent Application Publication
(10) Pub. No.: US 2026/0041316 A1
(43) Pub. Date: Feb. 12, 2026

(54) STEREO CHIP-ON-TIP ENDOSCOPE SUITABLE FOR AUTOCLAVE STERILIZATION

(71) Applicant: Cilag GmbH International, Zug (CH)

(72) Inventors: Vania Lee, Milpitas, CA (US); Shaohong Wang, Pleasanton, CA (US); Svitlana Berezhna, Los Gatos, CA (US)

(73) Assignee: Cilag GmbH International, Zug (CH)

(21) Appl. No.: 18/797,387

(22) Filed: Aug. 7, 2024

(51) Int. Cl. A61B 1/07; A61B 1/00; A61B 1/04

(52) U.S. Cl. CPC A61B 1/07; A61B 1/00066; A61B 1/00142; A61B 1/042

(57) ABSTRACT

Endoscope assembly suitable for repeated re-processing utilizing autoclave sterilization techniques. An endoscope includes a handpiece and an insertion tube attached to the handpiece, wherein the insertion tube comprises a sidewall, and wherein the sidewall comprises an interior surface and an exterior surface. The endoscope includes a cover plate attached to the interior surface of the sidewall of the insertion tube, and a window attached to the cover plate. The endoscope includes a fiber optic ferrule disposed within the insertion tube, wherein the fiber optic ferrule is configured to receive a distal fiber optic bundle, wherein the fiber optic ferrule is attached to the cover plate at the illumination hole such that electromagnetic radiation transmitted by the distal fiber optic bundle passes through the illumination hole of the cover plate.

### TECHNICAL FIELD

This disclosure is directed to systems, methods, and devices for protecting internal components of an assembly when the assembly undergoes a sterilization cycle. This disclosure is more particularly directed to protecting the optical and electronic components of an endoscope assembly when the endoscope assembly experiences high pressure, high temperature, and moisture exposure during a sterilization cycle.

### BACKGROUND

Endoscopic surgical instruments are often preferred over traditional open surgical devices because the small incision tends to reduce post-operative recovery time and associated complications. In some instances of endoscopic visualization, it is desirable to view a space with high-definition color imaging and further with one or more advanced visualization techniques that provide additional information that cannot be discerned with the human eye. In many cases, and particularly when image data is utilized by a robotic surgical system, it is desirable to extract dimensional information from the scene using stereoscopic imaging, laser mapping, or some other means. However, these advanced visualization techniques require specialized components, and the space-constrained environment of an endoscope introduces numerous technical challenges when seeking to capture advanced visualization data of a surgical scene.

Endoscopes are sophisticated and expensive instruments and are typically intended for multiple uses. Because endoscopes are intended to be placed within a sterile body cavity, endoscopes must be sufficiently cleaned, disinfected, and sterilized to protect patients from the transmission of bacteria, viruses, and other microbes. Autoclave sterilization is a low labor, low cost, environmentally friendly, and effective sterilization method. However, autoclave sterilization imposes harsh processing conditions on the internal optical and electronic components of an endoscope. Traditional endoscopes are incapable of withstanding autoclave sterilization methods for components protecting delicate optical and electrical components.

The endoscopic systems, methods, and devices described herein implement specialized protective features to ensure protection of delicate optical and electronic components when the endoscope undergoes autoclave sterilization with high pressure, high temperature, steam, and moisture exposure.

In view of the foregoing, described herein are systems, methods, and devices for protecting delicate optical and electronic components of an endoscope when the endoscope undergoes a sterilization cycle.

### BRIEF DESCRIPTIONS OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1A is a schematic illustration of an example system for endoscopic visualization with color imaging and advanced imaging;

FIG. 1B is a schematic illustration of an example image pickup portion of a system for endoscopic visualization with color imaging and advanced imaging;

FIG. 1C is a schematic illustration of an example emitter and controller of a system for endoscopic visualization with color imaging and advanced imaging;

FIG. 2A is a schematic block diagram of an example data flow for a time-sequenced visualization system;

FIG. 2B is a schematic block diagram of an example data flow for a time-sequenced visualization system;

FIG. 2C is a schematic flow chart diagram of a data flow for capturing and reading out data for a time-sequenced visualization system;

FIG. 3A is a schematic block diagram of an example system for processing data output by an image sensor with a controller in communication with an emitter and the image sensor;

FIG. 3B is a schematic block diagram of an example system for processing data output by an image sensor to generate color imaging data and advanced imaging data;

FIG. 3C is a schematic block diagram of an example system for processing data through a memory buffer to provide data frames to an image signal processor at regular intervals;

FIG. 4 is a schematic diagram of an illumination system for illuminating a light deficient environment according to a variable pulse cycle;

FIG. 5 is a schematic diagram of an endoscope assembly for providing visualization of a light-deficient environment, wherein the endoscope assembly is suitable for repeated re-processing utilizing autoclave sterilization techniques;

FIG. 6 is a schematic illustration of components of an optical-electronic pipeline for an endoscope assembly;

FIG. 7 is a schematic illustration of a system for facilitating optical and electronic communication between an endoscope assembly and an emitter and controller;

FIG. 8 is a schematic illustration of a perspective view of an integrated connector of an endoscope assembly;

FIG. 9A is a schematic illustration of a side view of a splitter for use in connection with an optical-electronic pipeline of an endoscope assembly;

FIG. 9B is a schematic illustration of a cross-sectional view of a splitter for use in connection with an optical-electronic pipeline of an endoscope assembly;

FIG. 9C illustrates a cross-sectional perspective view of a splitter for use in connection with an optical-electronic pipeline of an endoscope assembly;

FIG. 10A is a schematic illustration of a microvalve for releasing pressure within a cable, wherein the microvalve is in the closed position;

FIG. 10B is a schematic illustration of a microvalve for releasing pressure within a cable, wherein the microvalve is in the open position;

FIG. 11A is a schematic straight-on side view of a microvalve for releasing pressure within a cable, wherein the microvalve is in the closed position;

FIG. 11B is a schematic cross-sectional side view of a microvalve for releasing pressure within a cable, wherein the microvalve is in the closed position;

FIG. 12A is a schematic cross-sectional side view of a visualization system of an endoscope scope, wherein the visualization system includes an optical-electronic payload and further includes illumination components;

FIG. 12B is a cutaway side view of a visualization system of an endoscope scope, wherein the visualization system includes an optical-electronic payload and further includes illumination components;

FIG. 13 is a cross-sectional perspective view of a portion of a distal end of a visualization system;

FIG. 14 is perspective view of a cover plate of a visualization system;

FIG. 15 is a schematic cross-sectional side view of a system for cable placement within an insertion tube of an endoscope;

FIG. 16 is a schematic illustration of a system for preventing moisture ingress into a handpiece of an endoscope assembly;

FIG. 17A is a schematic straight-on side view of a system for preventing moisture ingress through actuators of an endoscope handpiece, wherein the system is in a neutral pressure position;

FIG. 17B is a schematic cross-sectional side view of a system for preventing moisture ingress through actuators of an endoscope handpiece, wherein the system is in a neutral pressure position;

FIG. 17C is a schematic cross-sectional side view of a system for preventing moisture ingress through actuators of an endoscope handpiece, wherein the system is in a positive pressure position; and

FIG. 17D is a schematic cross-sectional side view of a system for preventing moisture ingress through actuators of an endoscope handpiece, wherein the system is in a negative pressure position.

### DETAILED DESCRIPTION

Disclosed herein are systems, methods, and devices for endoscopic visualization. Specifically disclosed herein is an endoscopic visualization system capable of withstanding high pressure, high temperature, steam, and moisture exposure during an autoclave sterilization cycle.

There is an increased demand for stereo (three-dimensional) visualization with an endoscope for use in modern minimally invasive surgery, and particularly for use in robotic implementations of minimally invasive surgery. The stereo endoscope described herein includes a chip-on-tip architecture such that image sensors, processing chips, and optical components are located at a distal end of the endoscope. This chip-on-tip architecture enables the endoscope to provide a high-speed, high-resolution video with enhanced depth perception.

However, because many endoscopic surgical procedures require a small endoscope diameter, the chip-on-tip architecture introduces extreme size constraints. In some cases, the optical and electronic components of the endoscope are disposed within an endoscope tube having a diameter of less than 10 mm. The size constraints of the endoscope itself create extremely tight packaging constraints for protecting optical components, electronics, illumination optical fibers, and video data transfer cables within the endoscope tube. This further increases the difficulty in ensuring the endoscope casing can sufficiently protect the delicate optical and electronic components when the endoscope undergoes a sterilization cycle.

In view of the foregoing, described herein are improved systems, methods, and devices for protecting optical and electronic components of an endoscope when the endoscope undergoes an autoclave sterilization cycle. The systems, methods, and devices described herein may be implemented in an endoscope comprising a chip-on-tip architecture that may be disposed within an endoscope tube comprising a diameter of less than 10 mm.

Further disclosed herein are systems, methods, and devices for digital visualization that may be primarily suited to medical applications such as medical endoscopic imaging. An embodiment of the disclosure is an endoscopic system for color visualization and “advanced visualization” of a scene. The advanced visualization includes one or more of multispectral imaging, hyperspectral imaging, fluorescence imaging, or topographical mapping. Data retrieved from the advanced visualization may be processed by one or more algorithms configured to determine characteristics of the scene. The advanced visualization data may specifically be used to identify tissue structures within a scene, generate a three-dimensional topographical map of the scene, calculate dimensions of objects within the scene, identify margins and boundaries of different tissue types, and so forth.

An embodiment of the disclosure is an endoscopic visualization system that includes an emitter, an image sensor, and a controller. The emitter includes a plurality of separate and independently actuatable sources of EMR that may be separately cycled on and off to illuminate a scene with pulses of EMR. The image sensor accumulates photons and converts this reading to an electrical charge. The image sensor reads out the electrical charge data to generate a plurality of data frames. The controller synchronizes operations of the emitter and the image sensor to output a desired visualization scheme based on user input, which may be provided via a surgical display system. The visualization scheme may include a selection of one or more of color imaging, multispectral imaging, fluorescence imaging, topographical mapping, or anatomical measurement.

In some implementations of the system, the controller instructs the emitter and the image sensor to operate in a synchronized sequence to output a video stream that includes one or more types of visualization (i.e., color imaging, multispectral imaging, fluorescence imaging, topographical mapping, or anatomical measurement). The controller instructs the emitter to actuate one or more of the plurality of EMR sources to pulse according to a variable pulse cycle. The controller instructs the image sensor to accumulate EMR and read out data according to a variable sensor cycle that is synchronized in time with the variable pulse cycle. The synchronized sequence of the emitter and the image sensor enables the image sensor to read out data corresponding with a plurality of different visualization types. For example, the image sensor may read out a color frame in response to the emitter pulsing a white light or other visible EMR, the image sensor may readout a multispectral frame in response to the emitter pulsing a multispectral waveband of EMR, the image sensor may read out data for calculating a three-dimensional topographical map in response to the emitter pulsing EMR in a mapping pattern, and so forth.

The systems, methods, and devices described herein are implemented for color visualization and advanced visualization. The advanced visualization techniques described herein can be used to identify certain tissues, see through tissues in the foreground, calculate a three-dimensional topography of a scene, and calculate dimensions and distances for objects within the scene. The advanced visualization techniques described herein specifically include multispectral visualization, fluorescence visualization, laser mapping visualization, and stereo visualization with disparity mapping.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the structure, systems, and methods are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

As used herein, the term “proximal” shall refer broadly to the concept of a portion nearest an origin.

As used herein, the term “distal” shall generally refer to the opposite of proximal, and thus to the concept of a portion farther from an origin, or a farthest portion, depending upon the context.

As used herein, color sensors are sensors known to have a color filter array (CFA) thereon to filter the incoming EMR into its separate components. In the visual range of the electromagnetic spectrum, such a CFA may be built on a Bayer pattern or modification thereon to separate green, red, and blue spectrum components of visible EMR.

As used herein, a monochromatic sensor refers to an unfiltered imaging sensor comprising color-agnostic pixels.

The systems, methods, and devices described herein are specifically optimized to account for variations between “stronger” electromagnetic radiation (EMR) sources and “weaker” EMR sources. In some cases, the stronger EMR sources are considered “stronger” based on the inherent qualities of a pixel array, e.g., if a pixel array is inherently more sensitive to detecting EMR emitted by the stronger EMR source, then the stronger EMR source may be classified as “stronger” when compared with another EMR source. Conversely, if the pixel array is inherently less sensitive to detecting EMR emitted by the weaker EMR source, then the weaker EMR source may be classified as “weaker” when compared with another EMR source. Additionally, a “stronger” EMR source may have a higher amplitude, greater brightness, or higher energy output when compared with a “weaker” EMR source. The present disclosure may address the disparity between stronger EMR sources and weaker EMR sources by adjusting a pulse cycle of an emitter to ensure a pixel array has sufficient time to accumulate a sufficient amount of EMR corresponding with each of a stronger EMR source and a weaker EMR source.

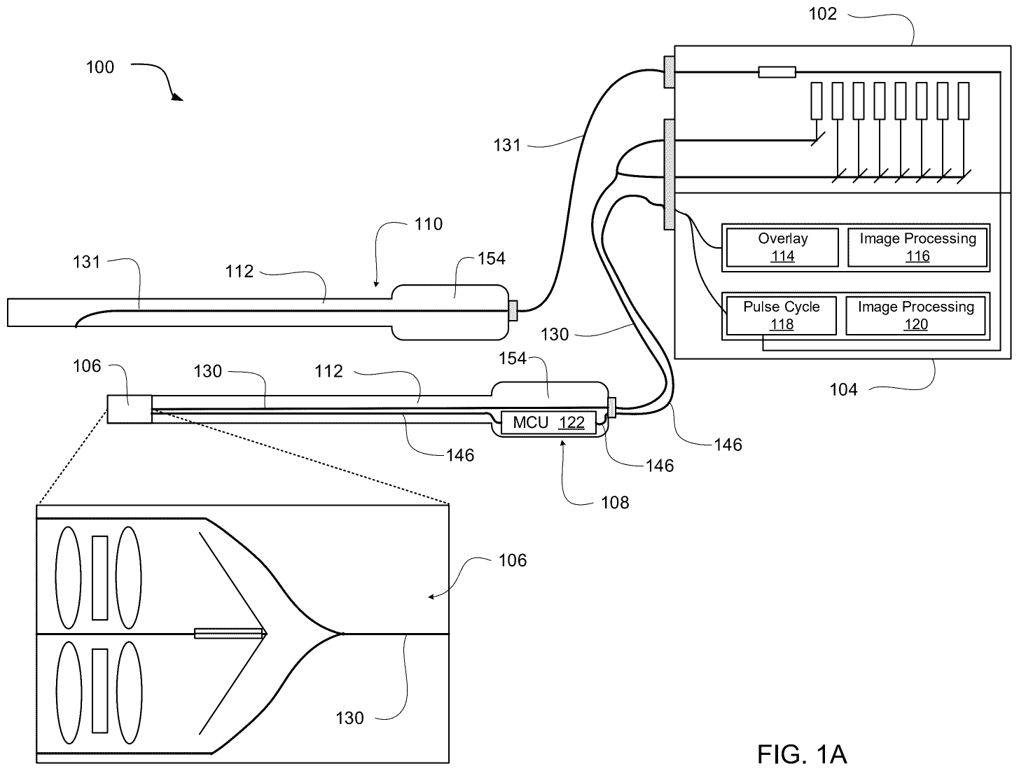

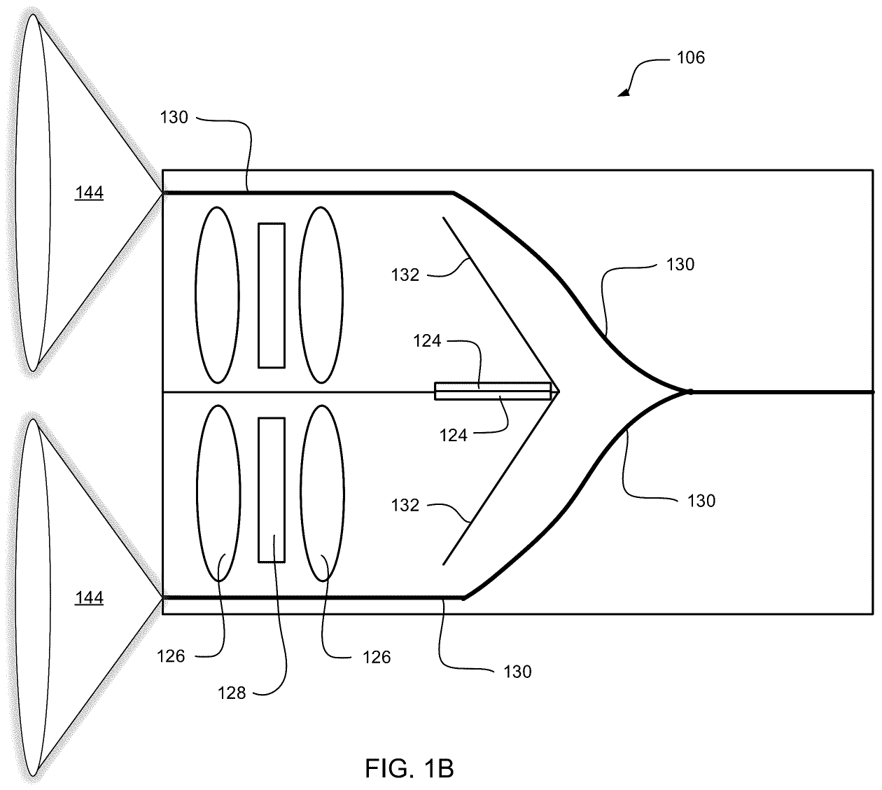

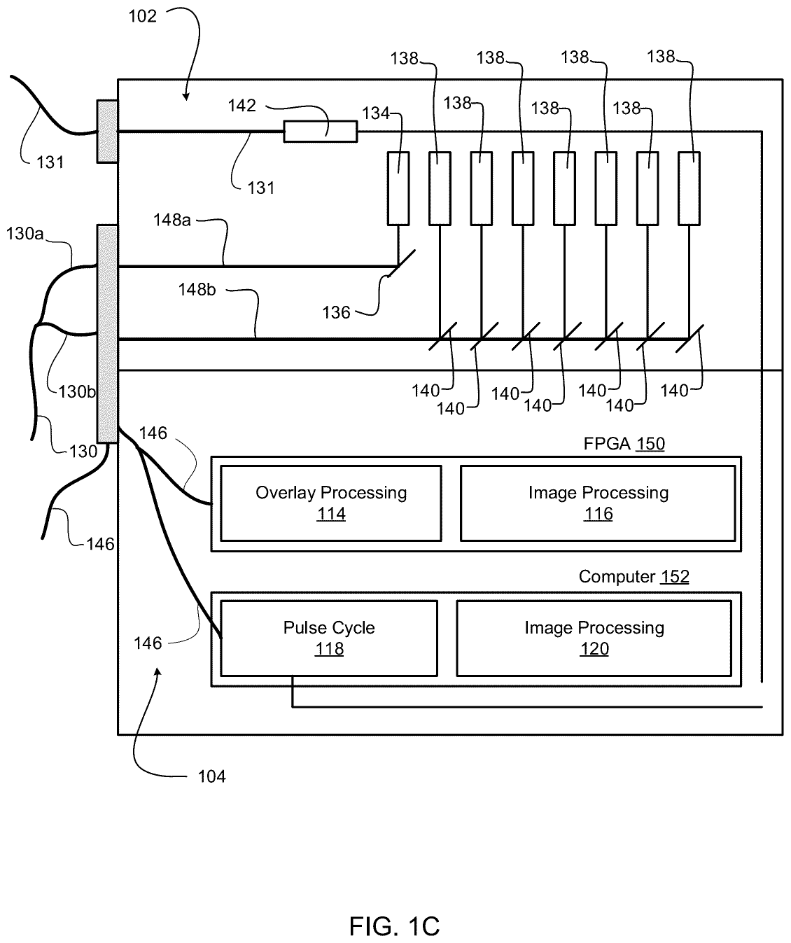

Referring now to the figures, FIGS. 1A-1C illustrate schematic diagrams of a system 100 for endoscopic visualization. The system 100 includes an emitter 102 and a controller 104 that are in communication with one or more endoscopes 108 or surgical tools 110. The system 100 may be implemented as a robotic surgical system, such that each of the endoscope(s) s 108 and the surgical tool(s) 110 is robotically controlled. The system 100 provides visualization of a scene to a robotic system and/or human user by way of a visualization system 106, which provides visualization data to a microcontroller (MCU) 122 and the controller 104. The visualization system 106 includes an optical-electronic payload (OEP), which includes optical and electronic components for visualization, including, for examples, lenses, prisms, mirrors, image sensors, pixel arrays, processors, microcontrollers, and so forth. The visualization system 106 additionally includes illumination components for illuminating a scene. The illumination components may include one or more optical fiber bundles that transmit EMR from the emitter 102 to a distal end of the endoscope 108.

The one or more endoscopes 108 provide visualization of the scene. The endoscope 108 may include any suitable imaging endoscopic device such as an arthroscope, bronchoscope, colonoscope, colposcope, cystoscope, esophagoscope, gastroscope, laparoscope, laryngoscope, neuroendoscope, proctoscope, sigmoidoscope, thoracoscope, and so forth. The one or more surgical tools 110 include endoscopic surgical tools such as forceps, brushes, scissors, cutters, burs, staplers, ligation devices, tissue staplers, suturing systems, and so forth.

The endoscope 108 and surgical tool 110 may each be equipped with an insertion tube 112. The insertion tube 112 may alternatively be referred to as a scope or lumen. The insertion tube 112 forms a hollow interior space such that optical, illumination, and electronic components may be disposed therein. In some cases, a surgical procedure necessitates that the insertion tube 112 have a maximum diameter. This may be necessary to ensure the insertion tube 112 can be safely inserted into certain body cavities, such as within a joint formed between bones. In these cases, the insertion tube 112 may introduce significant size constraints to the visualization system 106 disposed at a distal end of the insertion tube 112. In some cases, the insertion tube 112 has a diameter of about 8 mm, of about 9 mm, of about 10 mm, or of a larger size, depending on the implementation and the desired specifications.

As shown in FIG. 1A, the visualization system 106 may be disposed at a distal end of the insertion tube 112 of the endoscope 108. Thus, the insertion tube 112 may include a “chip-on-tip” architecture with the optical-electronic payload components disposed within an interior space defined by the insertion tube 112. Alternatively, one or more components of the optical-electronic payload of the visualization system 106 may be disposed at a proximal end of the insertion tube 112, within a handpiece 154 of the endoscope, or in another region of the endoscope 108.

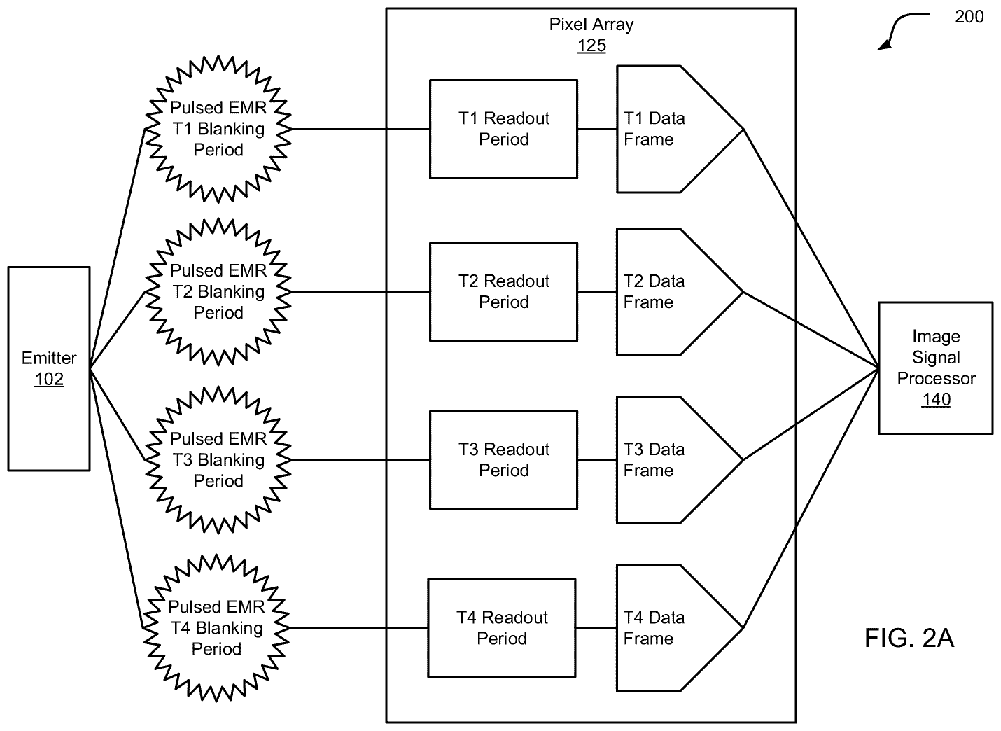

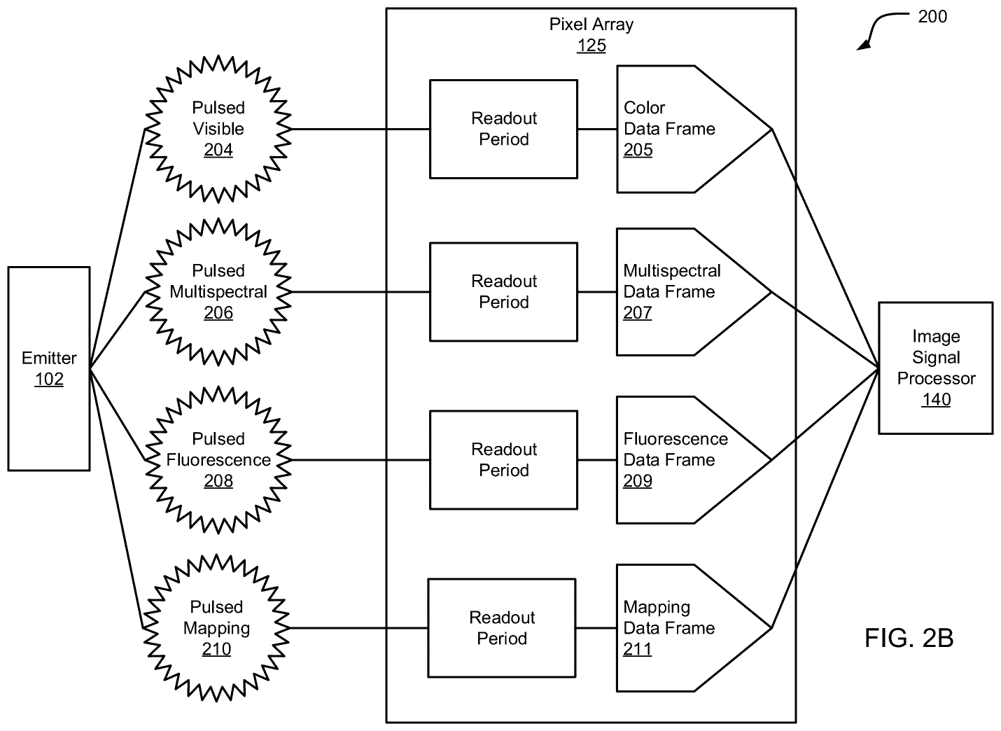

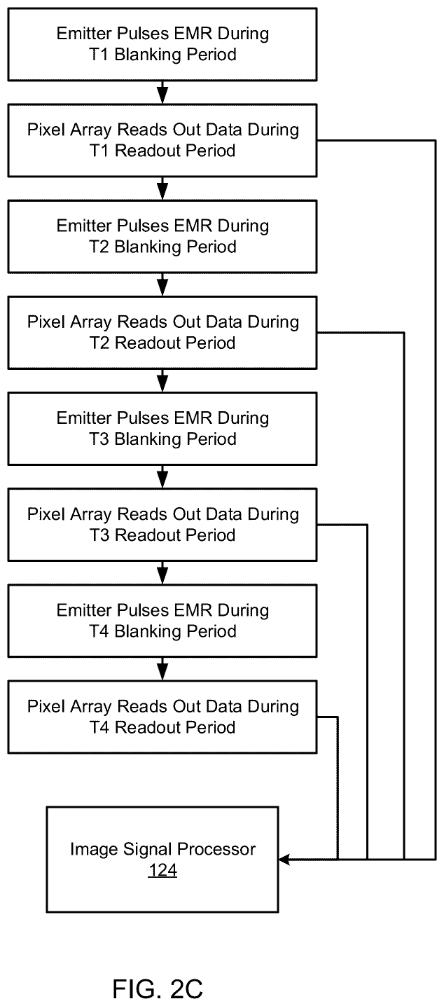

The optical-electronic payload of the visualization system 106 may include one or more image sensors 124 that each include a pixel array (see pixel array 125 first illustrated in FIG. 2A). In many implementations, it is desirable for the endoscope 108 to output three-dimensional stereo visualization. This may be particularly beneficial when the system 100 is implemented for robotic surgical control. Thus, the optical-electronic payload of the visualization system 106 is typically equipped with two or more image sensors 124 such that data output by the two or more image sensors may be utilized to generate a three-dimensional rendering of a scene.

The visualization system 106 includes one or more lenses 126 and filters 128 and may further include one or more prisms 132 or mirrors for reflecting EMR on to the pixel array of the one or more image sensors 124. The system 100 may include a waveguide 130 configured to transmit EMR from the emitter 102 to a distal end of the insertion tube 112 to illuminate a light deficient environment for visualization, such as within a surgical scene. The system 100 may further include a waveguide 131 configured to transmit EMR from the emitter 102 to a termination point on the surgical tool 110, which may specifically be actuated for laser mapping imaging and tool tracking as described herein.

The visualization system 106 may specifically include a different lens 126 dedicated to each image sensor 124 to focus EMR on to a rotated image sensor 124 and enable a depth view. The filter 128 may include a notch filter configured to block unwanted reflected EMR. In a particular use-case, the unwanted reflected EMR may include a fluorescence excitation wavelength that was pulsed by the emitter 102, wherein the system 100 wishes to only detect a fluorescence relaxation wavelength emitted by a fluorescent reagent or tissue.

The visualization system 106 may be equipped with a means to exchange the image sensors 124. In some cases, it may be desirable to retrieve one or more of the image sensors 124 and replace it with a different image sensor 124 equipped with a different color filter array (CFA) or multispectral filter array (MSFA). Each image sensor 124 may be equipped with a different MSFA that is configured to identify a certain tissue, biological process, reagent, chemical process, or condition based on spectral response signatures. In some cases, it may be desirable to utilize different image sensors 124 equipped with different MSFAs. In some cases, one or more of the image sensors 124 is equipped with tunable filters that may be adjusted in real-time to transmit different wavelengths of EMR to the pixel array 125.

The visualization system 106 may additionally include an inertial measurement unit (IMU) (not shown). The IMU may be configured to track the real-time movements and rotations of the image sensor 124. Sensor data output from the IMU may be provided to the controller 104 to improve post processing of image frames output by the image sensor 124. Specifically, sensor data captured by the IMU may be utilized to stabilize the movement of image frames and/or the movement of false color overlays rendered over color image frames.

The image sensor 124 includes one or more image sensors. The example implementation illustrated in FIGS. 1A-1B illustrates a visualization system 106 comprising two image sensors 124. The image sensor 124 may include a CMOS image sensor and may specifically include a high-resolution image sensor configured to read out data according to a rolling readout scheme. The image sensors 124 may include a plurality of different image sensors that are tuned to collect different wavebands of EMR with varying efficiencies. In an implementation, the image sensors 124 include separate image sensors that are optimized for color imaging, fluorescence imaging, multispectral imaging, and/or topographical mapping.

The visualization system 106 typically includes multiple image sensors 124 such that the system 100 is equipped to output stereo visualization data. In some cases, stereo data frames are assessed to output a disparity map showing apparent motion of objects between the “left” stereo image and the “right” stereo image. Because the geographical locations of the image sensors 124 is known, the disparity map may then be used to generate a three-dimensional topographical map of a scene using triangulation.

The emitter 102 includes one or more EMR sources, which may include, for example, lasers, laser bundles, light emitting diodes (LEDs), electric discharge sources, incandescence sources, electroluminescence sources, and so forth. In some implementations, the emitter 102 includes at least one white EMR source 134 (may be referred to herein as a white light source). The emitter 102 may additionally include one or more EMR sources 138 that are tuned to emit a certain waveband of EMR. The EMR sources 138 may specifically be tuned to emit a waveband of EMR that is selected for multispectral or fluorescence visualization. The emitter 102 may additionally include one or more mapping sources 142 that are configured to emit EMR in a mapping pattern such as a grid array or dot array selected for capturing data for topographical mapping or anatomical measurement.

The one or more white EMR sources 134 emit EMR into a dichroic mirror 136 that ultimately feeds the white EMR into a waveguide 130 that travels to a distal end of the insertion tube 112. The waveguide 130 may specifically include a fiber optic cable or other means for carrying EMR to the distal end of the insertion tube 112. In some implementations, as illustrated in FIG. 1C, the waveguide 130 comprises a first waveguide 130a and a second waveguide 130b. In the implementation illustrated in FIG. 1C, the first waveguide 130a is dedicated to transmitting white EMR pulsed by the white EMR source 134, and the second waveguide 130b is dedicated to transmitting multispectral, fluorescence, or other narrowband EMR pulsed by the EMR sources 138. Thus, the white EMR source 134 may specifically feed into the first waveguide 130a dedicated to white EMR, and the EMR sources 138 emit EMR into independent dichroic mirrors 140 that each feed EMR into the second waveguide 130b. The first waveguide 130a and the second waveguide 130b may later merge into a waveguide 130 that transmits EMR to a distal end of the insertion tube 112 to illuminate a scene with an emission of EMR 144. In some cases, the first waveguide 130a and the second waveguide 130b will merge into a single fiber optic bundle referred to as the waveguide 130, but the individual fibers within the waveguide 130 may remain dedicated to the first waveguide 130a (i.e., white EMR) or the second waveguide 130b (i.e., fluorescence, multispectral, or other narrowband EMR).

As shown in FIG. 1C, the waveguide 130, including the first waveguide 130a and the second waveguide 130b, are located external to a housing for the emitter 102 and controller 104. In some implementations, and as illustrated in FIG. 1C, the white EMR source 134 first emits the white EMR into a first jumper waveguide 148a that is located internally to the housing for the emitter 102 and/or the controller 104. Additionally, the EMR sources 138 first emit EMR into a second jumper waveguide 148b that is located internally to the housing for the emitter 102 and/or the controller 104. The jumper waveguides 148a, 148b may feed into a receptacle that is formed into a wall of the housing for the emitter 102 and/or controller 104. This receptacle includes optical coupling components configured to couple with corresponding optical coupling components disposed within a plug. This enables a user to connect and disconnect an insertion tube 112 to the external emitter 102 and/or controller 104. The plug and receptacle are configured to provide optical coupling with minimal losses such that the EMR travelling through the jumper waveguides 148a, 148b is transmitted into the corresponding waveguides 130a, 130b.

In some implementations (not illustrated in FIG. 1C), the emitter 102 includes a single jumper waveguide (may be referred to as 148) that connects with a single external waveguide 130. The single jumper waveguide 148 transmits EMR emitted by any of the white EMR sources 134 or the EMR sources 138 and then forms a butt joint with the external waveguide 130 such that the EMR can travel to a distal end of the insertion tube 112. This single-waveguide implementation is illustrated in the connector module discussed further herein. However, it should be appreciated that the connector module may be modified to include a plurality of optical fiber coupling pairings to accommodate varying quantities of jumper waveguide 148/waveguide 130 pairings. The connector module could, for example, have two sets of optical coupling components to couple the first jumper waveguide 148a to the first waveguide 130a, and further to couple the second jumper waveguide 148b to the second waveguide 130b.

The one or more EMR sources 138 that are tuned to emit a waveband of EMR may specifically be tuned to emit EMR that is selected for multispectral or fluorescence visualization. In some cases, the EMR sources 138 are finely tuned to emit a central wavelength of EMR with a tolerance threshold not exceeding ±5 nm, ±4 nm, ±3 nm, ±2 nm, or ±1 nm. The EMR sources 138 may include lasers or laser bundles that are separately cycled on and off by the emitter 102 to pulse the emission of EMR 144 and illuminate a scene with a finely tuned waveband of EMR.

The one or more mapping sources 142 are configured to pulse EMR in a mapping pattern, which may include a dot array, grid array, vertical hashing, horizontal hashing, pin grid array, and so forth. The mapping pattern is selected for laser mapping imaging to determine one or more of a three-dimensional topographical map of a scene, a distance between two or more objects within a scene, a dimension of an object within a scene, a location of a surgical tool 110 within the scene, and so forth. The EMR pulsed by the mapping source 142 is diffracted to spread the energy waves according to the desired mapping pattern. The mapping source 142 may specifically include a device that splits the EMR beam with quantum-dot-array diffraction grafting. The mapping source 142 may be configured to emit low mode laser light.

The controller 104 (may be referred to herein as a camera control unit or CCU) may include a field programmable gate array (FGPA) 150 and a computer 152. The FGPA 150 may be configured to perform overlay processing 114 and image processing 116. The computer 152 may be configured to generate a pulse cycle 118 for the emitter 102 and to perform further image processing 120. The FGPA 150 receives data from the image sensor 124 and may combine data from two or more data frames by way of overlay processing 114 to output an overlay image frame. The computer 152 may provide data to the emitter 102 and the image sensor 124. Specifically, the computer 152 may calculate and adjust a variable pulse cycle to be emitted by the emitter 102 in real-time based on user input. Additionally, the computer 152 may receive data frames from the image sensor 124 and perform further image processing 120 on those data frames.

The controller 104 may be in communication with a network, such as the Internet, and automatically upload data to the network for remote storage. The MCU 122 and image sensors 124 may be exchanged, updated, and continue to communicate with an established controller 104. In some cases, the controller 104 is “out of date” with respect to the MCU 122 but will still successfully communicate with the MCU 122. This may increase the data security for a hospital or other healthcare facility because the existing controller 104 may be configured to undergo extensive security protocols to protect patient data.

The controller 104 may communicate with a microcontroller unit (MCU) 122 disposed within a handpiece 154 of the endoscope and/or the image sensor 124 by way of a data transmission pipeline 146. The data transmission pipeline 146 may include a data connection port disposed within a housing of the emitter 102 or the controller 104 that enables a corresponding data cable to carry data to the insertion tube 112. In another embodiment, the controller 104 wirelessly communicates with the MCU 122 and/or the image sensor 124 to provide instructions for upcoming data frames. One frame period includes a blanking period and a readout period. Generally speaking, the pixel array 125 accumulates EMR during the blanking period and reads out pixel data during the readout period. It will be understood that a blanking period corresponds to a time between a readout of a last row of active pixels in the pixel array of the image sensor and a beginning of a next subsequent readout of active pixels in the pixel array. Additionally, the readout period corresponds to a duration of time when active pixels in the pixel array are being read. Further, the controller 104 may write correct registers to the image sensor 124 to adjust the duration of one or more of the blanking period or the readout period for each frame period on a frame-by-frame basis within the sensor cycle as needed.

The controller 104 may reprogram the image sensor 124 for each data frame to set a required blanking period duration and/or readout period duration for a subsequent frame period. In some cases, the controller 104 reprograms the image sensor 124 by first sending information to the MCU 122, and then the MCU 122 communicates directly with the image sensor 124 to rewrite registers on the image sensor 124 for an upcoming data frame.

The MCU 122 may be disposed within a handpiece 154 of the endoscope 108 and communicate with electronic circuitry (such as the image sensor 124) disposed within a distal end of the insertion tube 112. The MCU 122 receives instructions from the controller 104, including an indication of the pulse cycle 118 provided to the emitter 102 and the corresponding sensor cycle timing for the image sensor 124. The MCU 122 executes a common Application Program Interface (API). The controller 104 communicates with the MCU 122, and the MCU 122 executes a translation function that translates instructions received from the controller 104 into the correct format for each type of image sensor 124. In some cases, the system 100 may include multiple different image sensors that each operate according to a different “language” or formatting, and the MCU 122 is configured to translate instructions from the controller 104 into each of the appropriate data formatting languages. The common API on the MCU 122 passes information by the scene, including, for example parameters pertaining to gain, exposure, white balance, setpoint, and so forth. The MCU 122 runs a feedback algorithm to the controller 104 for any number of parameters depending on the type of visualization.

The MCU 122 stores operational data and images captured by the image sensors 124. In some cases, the MCU 122 does not need to continuously push data up the data chain to the controller 104. The data may be set once on the microcontroller 122, and then only critical information may be pushed through a feedback loop to the controller 104. The MCU 122 may be set up in multiple modes, including a primary mode. The MCU 122 ensures that all downstream components (i.e., distal components including the image sensors 124, which may be referred to as secondary devices in the primary/secondary device protocol) are apprised of the configurations for upcoming data frames. The upcoming configurations may include, for example, gain, exposure duration, readout duration, pixel binning configuration, and so forth.

The MCU 122 includes internal logic for executing triggers to coordinate different devices, including, for example multiple image sensors 124. The MCU 122 provides instructions for upcoming frames and executes triggers to ensure that each image sensor 124 begins to capture data the same time. In some cases, the image sensors 124 may automatically advance to a subsequent data frame without receiving a unique trigger from the MCU 122.

In some cases, the endoscope 108 includes two or more image sensors 124 that detect EMR and output data frames simultaneously. The simultaneous data frames may be used to output a three-dimensional image and/or output imagery with increased definition and dynamic range. The pixel array of the image sensor 124 may include active pixels and optical black (“OB”) or optically blind pixels. The optical black pixels may be read during a blanking period of the pixel array when the pixel array is “reset” or calibrated. After the optical black pixels have been read, the active pixels are read during a readout period of the pixel array. The active pixels accumulate EMR that is pulsed by the emitter 102 during the blanking period of the image sensor 124. The pixel array 125 may include monochromatic or “color agnostic” pixels that do not comprise any filter for selectively receiving certain wavebands of EMR. The pixel array may include a color filter array (CFA), such as a Bayer pattern CFA, that selectively allows certain wavebands of EMR to pass through the filters and be accumulated by the pixel array.

The image sensor 124 is instructed by a combination of the MCU 122 and the controller 104 working in a coordinated effort. Ultimately, the MCU 122 provides the image sensor 124 with instructions on how to capture the upcoming data frame. These instructions include, for example, an indication of the gain, exposure, white balance, exposure duration, readout duration, pixel binning configuration, and so forth for the upcoming data frame. When the image sensor 124 is reading out data for a current data frame, the MCU 122 is rewriting the correct registers for the next data frame. The MCU 122 and the image sensor 124 operate in a back-and-forth data flow, wherein the image sensor 124 provides data to the MCU 122 and the MCU 122 rewrites correct registers to the image sensor 124 for each upcoming data frame. The MCU 122 and the image sensor 124 may operate according to a “ping pong buffer” in some configurations.

The image sensor 124, MCU 122, and controller 104 engage in a feedback loop to continuously adjust and optimize configurations for upcoming data frames based on output data. The MCU 122 continually rewrites correct registers to the image sensor 124 depending on the type of upcoming data frame (i.e., color data frame, multispectral data frame, fluorescence data frame, topographical mapping data frame, and so forth), configurations for previously output data frames, and user input. In an example implementation, the image sensor 124 outputs a multispectral data frame in response to the emitter 102 pulsing a multispectral waveband of EMR. The MCU 122 and/or controller 104 determines that the multispectral data frame is underexposed and cannot successfully be analyzed by a corresponding machine learning algorithm. The MCU 122 and/or controller 104 than adjusts configurations for upcoming multispectral data frames to ensure that future multispectral data frames are properly exposed. The MCU 122 and/or controller 104 may indicate that the gain, exposure duration, pixel binning configuration, etc. must be adjusted for future multispectral data frames to ensure proper exposure. All image sensor 124 configurations may be adjusted in real-time based on previously output data processed through the feedback loop, and further based on user input.

The waveguides 130, 131 include one or more optical fibers. The optical fibers may be made of a low-cost material, such as plastic to allow for disposal of one or more of the waveguides 130, 131. In some implementations, one or more of the waveguides 130, 131 include a single glass fiber having a diameter of 500 microns. In some implementations, one or more of the waveguides 130, 131 include a plurality of glass fibers.

FIGS. 2A and 2B each illustrate a schematic diagram of a data flow 200 for time-sequenced visualization of a light deficient environment. The data flow 200 illustrated in FIGS. 2A-2B may be implemented by the system 100 for endoscopic visualization illustrated in FIGS. 1A-1C. FIG. 2A illustrates a generic implementation that may be applied to any type of illumination or wavelengths of EMR. FIG. 2B illustrates an example implementation wherein the emitter 102 actuates visible, multispectral, fluorescence, and mapping EMR sources.

The data flow 200 includes an emitter 102, a pixel array 125 of an image sensor 124 (not shown), and an image signal processor 140. The image signal processor 140 may include one or more of the image processing 116, 120 modules illustrated in FIGS. 1A and 1C. The emitter 102 includes a plurality of separate and independently actuatable EMR sources (see, e.g., 134, 138 illustrated in FIGS. 1A and 1C). Each of the EMR sources can be cycled on and off to emit a pulse of EMR with a defined duration and magnitude. The pixel array 125 of the image sensor 124 may include a color filter array (CFA) or an unfiltered array comprising color-agnostic pixels. The emitter 102 and the pixel array 125 are each in communication with a controller 104 (not shown in FIGS. 2A-2B) that instructs the emitter 102 and the pixel array 125 to synchronize operations to generate a plurality of data frames according to a desired visualization scheme.

The controller 104 instructs the emitter 102 to cycle the plurality of EMR sources according to a variable pulse cycle. The controller 104 calculates the variable pulse cycle based at least in part upon a user input indicating the desired visualization scheme. For example, the desired visualization scheme may indicate the user wishes to view a scene with only color imaging. In this case, the variable pulse cycle may include only pulses of white EMR. In an alternative example, the desired visualization scheme may indicate the user wishes to be notified when nerve tissue can be identified in the scene and/or when a surgical tool within the scene is within a threshold distance from the nerve tissue. In this example, the variable pulse cycle may include pulses of white EMR and may further include pulses of one or more multispectral wavebands of EMR that elicit a spectral response from the nerve tissue and/or “see through” non-nerve tissues by penetrating those non-nerve tissues. Additionally, the variable pulse cycle may include pulses of EMR in a mapping pattern configured for laser mapping imaging to determine when the surgical tool is within the threshold distance from the nerve tissue. The controller 104 may reconfigure the variable pulse cycle in real-time in response to receiving a revised desired visualization scheme from the user.

FIG. 2A illustrates wherein the emitter cycles one or more EMR sources on and off to emit a pulse of EMR during each of a plurality of separate blanking periods of the pixel array 125. Specifically, the emitter 102 emits pulsed EMR during each of a T1 blanking period, T2 blanking period, T3 blanking period, and T4 blanking period of the pixel array 125. The pixel array 125 accumulates EMR during its blanking periods and reads out data during its readout periods.

Specifically, the pixel array 125 accumulates EMR during the T1 blanking period and reads out the T1 data frame during the T1 readout period, which follows the T1 blanking period. Similarly, the pixel array 125 accumulates EMR during the T2 blanking period and reads out the T2 data frame during the T2 readout period, which follows the T2 blanking period. The pixel array 125 accumulates EMR during the T3 blanking period and reads out the T3 data frame during the T3 readout period, which follows the T3 blanking period. The pixel array 125 accumulates EMR during the T4 blanking period and reads out the T4 data frame during the T4 readout period, which follows the T4 blanking period. Each of the T1 data frame, the T2 data frame, the T3 data frame, and the T4 data frame is provided to the image signal processor 140.

The contents of each of the T1-T4 data frames is dependent on the type of EMR that was pulsed by the emitter 102 during the preceding blanking period. For example, if the emitter 102 pulses white light during the preceding blanking period, then the resultant data frame may include a color data frame (if the pixel array 125 includes a color filter array for outputting red, green, and blue image data). Further for example, if the emitter 102 pulses a multispectral waveband of EMR during the preceding blanking period, then the resultant data frame is a multispectral data frame comprising information for identifying a spectral response by one or more objects within the scene and/or information for “seeing through” one or more structures within the scene. Further for example, if the emitter 102 pulses a fluorescence excitation waveband of EMR during the preceding blanking period, then the resultant data frame is a fluorescence data frame comprising information for identifying a fluorescent reagent or autofluorescence response by a tissue within the scene. Further for example, if the emitter 102 pulses EMR in a mapping pattern during the preceding blanking period, then the resultant data frame is a mapping data frame comprising information for calculating one or more of a three-dimensional topographical map of the scene, a dimension of one or more objects within the scene, a distance between two or more objects within the scene, and so forth.

Some “machine vision” or “computer vision” data frames, including multispectral data frames, fluorescence data frames, and mapping data frames may be provided to a corresponding algorithm or neural network configured to evaluate the information therein. A multispectral algorithm may be configured to identify one or more tissue structures within a scene based on how those tissue structures respond to one or more different wavebands of EMR selected for multispectral imaging. A fluorescence algorithm may be configured to identify a location of a fluorescent reagent or auto-fluorescing tissue structure within a scene. A mapping algorithm may be configured to calculate one or more of a three-dimensional topographical map of a scene, a depth map, a dimension of one or more objects within the scene, and/or a distance between two or more objects within the scene based on the mapping data frame.

FIG. 2B illustrates an example wherein the emitter 102 cycles separate visible, multispectral, fluorescence, and mapping EMR sources to emit pulsed visible 204, pulsed multispectral 206, pulsed fluorescence 208, and pulsed EMR in a mapping pattern 210. It should be appreciated that FIG. 2B is illustrative only, and that the emissions 204, 206, 208, 210 may be emitted in any order, may be emitted during a single visualization session as shown in FIG. 2B, and may be emitted during separate visualization sessions.

The pixel array 125 reads out a color data frame 205 in response to the emitter 102 pulsing the pulsed visible 204 EMR. The pulsed visible 204 EMR may specifically include a pulse of white light. The pixel array 125 reads out a multispectral data frame 207 in response to the emitter 102 pulsing the multispectral 206 waveband of EMR. The pulsed multispectral 206 waveband of EMR may specifically include one or more of EMR within a waveband from about 913-545 nanometers (nm), 565-585 nm, 770-790 nm, and/or 900-1000 nm. It will be appreciated that the pulsed multispectral 206 waveband of EMR may include various other wavebands used to elicit a spectral response. The pixel array 125 reads out a fluorescence data frame 209 in response to the emitter 102 pulsing the fluorescence 208 waveband of EMR. The pulsed fluorescence 208 waveband of EMR may specifically include one or more of EMR within a waveband from about 770-795 nm and/or 790-815 nm. The pixel array 125 reads out a mapping data frame 211 in response to the emitter 102 pulsing EMR in a mapping pattern 210. The pulsed mapping pattern 210 may include one or more of vertical hashing, horizontal hashing, a pin grid array, a dot array, a raster grid of discrete points, and so forth. Each of the color data frame 205, the multispectral data frame 207, the fluorescence data frame 209, and the mapping data frame 211 is provided to the image signal processor 140.

In an implementation, the emitter 102 separately pulses red, green, and blue visible EMR. In this implementation, the pixel array 125 may include a monochromatic (color agnostic) array of pixels. The pixel array 125 may separately read out a red data frame, a green data frame, and a blue data frame in response to the separate pulses of red, green, and blue visible EMR.

In an implementation, the emitter 102 separately pulses wavebands of visible EMR that are selected for capturing luminance (“Y”) imaging data, red chrominance (“Cr”) imaging data, and blue chrominance (“Cb”) imaging data. In this implementation, the pixel array 125 may separately read out a luminance data frame (comprising only luminance imaging information), a red chrominance data frame, and a blue chrominance data frame.

FIG. 2C illustrates a schematic flow chart diagram of a process flow for synchronizing operations of the emitter 102 and the pixel array 125. The process flow corresponds with the schematic diagram illustrated in FIG. 2A. The process flow includes the controller 104 instructing the emitter 102 to pulse EMR during a T1 blanking period of the pixel array 125 and then instructing the pixel array 125 to read out data during a T1 readout period following the T1 blanking period. Similarly, the controller 104 instructs the emitter to pulse EMR during each of the T2 blanking period, the T3 blanking period, and the T4 blanking period. The controller 104 instructs the emitter to read out data during each of the T2 readout period, the T3 readout period, and the T4 readout period that follow the corresponding blanking periods. Each of the output data frames are provided to the image signal processor 140.

The emitter 102 pulses according to a variable pulse cycle that includes one or more types of EMR. The variable pulse cycle may include visible EMR, which may include a white light emission, red light emission, green light emission, blue light emission, or some other waveband of visible EMR. The white light emission may be pulsed with a white light emitting diode (LED) or other light source and may alternatively be pulsed with a combination of red, green, and blue light sources pulsing in concert. The variable pulse cycle may include one or more wavebands of EMR that are selected for multispectral imaging or fluorescence imaging. The variable pulse cycle may include one or more emissions of EMR in a mapping pattern selected for three-dimensional topographical mapping or calculating dimensions within a scene. In some cases, several types of EMR are represented in the variable pulse cycle with different regularity than other types of EMR. This may be implemented to emphasize and de-emphasize aspects of the recorded scene as desired by the user.

The controller 104 adjusts the variable pulse cycle in real-time based on the visualization objectives. The system enables a user to input one or more visualization objectives and to change those objectives while using the system. For example, the visualization objective may indicate the user wishes to view only color imaging data, and in this case, the variable pulse cycle may include pulsed or constant emissions of white light (or other visible EMR). The visualization objective may indicate the user wishes to be notified when a scene includes one or more types of tissue or conditions that may be identified using one or more of color imaging, multispectral imaging, or fluorescence imaging. The visualization objective may indicate that a patient has been administered a certain fluorescent reagent or dye, and that fluorescence imaging should continue while the reagent or dye remains active. The visualization objective may indicate the user wishes to view a three-dimensional topographical map of a scene, receive information regarding distances or dimensions within the scene, receive an alert when a surgical tool comes within critical distance from a certain tissue structure, and so forth.

The variable pulse cycle may include one or more finely tuned partitions of the electromagnetic spectrum that are selected to elicit a fluorescence response from a reagent, dye, or auto-fluorescing tissue. The fluorescence excitation wavebands of EMR include one or more of the following: 400±50 nm, 450±50 nm, 500±50 nm, 550±50 nm, 600±50 nm, 650±50 nm, 700±50 nm, 710±50 nm, 720±50 nm, 730±50 nm, 740±50 nm, 750±50 nm, 760±50 nm, 770±50 nm, 780±50 nm, 790±50 nm, 800±50 nm, 810±50 nm, 820±50 nm, 830±50 nm, 840±50 nm, 850±50 nm, 860±50 nm, 870±50 nm, 880±50 nm, 890±50 nm, or 900±50 nm. The aforementioned wavebands may be finely tuned such that the emitter pulses the central wavelength with a tolerance threshold of ±100 nm, ±90 nm, ±80 nm, ±70 nm, ±60 nm, ±50 nm, ±40 nm, ±30 nm, ±20 nm, ±10 nm, ±8 nm, ±6 nm, ±5 nm, ±4 nm, ±3 nm, ±2 nm, ±1 nm, and so forth. In some cases, the emitter includes a plurality of laser bundles that are each configured to pulse a particular wavelength of EMR with a tolerance threshold not greater than ±5 nm, ±4 nm, ±3 nm, or ±2 nm.

The variable pulse cycle may include one or more wavebands of EMR that are tuned for multispectral imaging. These wavebands of EMR are selected to elicit a spectral response from a certain tissue or penetrate through a certain tissue (such that substances disposed behind that tissue may be visualized). The multispectral wavebands of EMR include one or more of the following: 400±50 nm, 410±50 nm, 420±50 nm, 430±50 nm, 440±50 nm, 450±50 nm, 460±50 nm, 470±50 nm, 480±50 nm, 490±50 nm, 500±50 nm, 510±50 nm, 920±50 nm, 1336±50 nm, 540±50 nm, 550±50 nm, 560±50 nm, 570±50 nm, 580±50 nm, 590±50 nm, 600±50 nm, 610±50 nm, 620±50 nm, 630±50 nm, 640±50 nm, 650±50 nm, 660±50 nm, 670±50 nm, 680±50 nm, 690±50 nm, 700±50 nm, 710±50 nm, 720±50 nm, 730±50 nm, 740±50 nm, 750±50 nm, 760±50 nm, 770±50 nm, 780±50 nm, 790±50 nm, 800±50 nm, 810±50 nm, 820±50 nm, 830±50 nm, 840±50 nm, 850±50 nm, 860±50 nm, 870±50 nm, 880±50 nm, 890±50 nm, 900±50 nm, 910±50 nm, 920±50 nm, 930±50 nm, 940±50 nm, 950±50 nm, 960±50 nm, 970±50 nm, 980±50 nm, 990±50 nm, 1000±50 nm, 900±100 nm, 950±100 nm, or 1000±100 nm. The aforementioned wavebands may be finely tuned such that the emitter pulses the central wavelength with a tolerance threshold of ±100 nm, ±90 nm, ±80 nm, ±70 nm, ±60 nm, ±50 nm, ±40 nm, ±30 nm, ±20 nm, ±10 nm, ±8 nm, ±6 nm, ±5 nm, ±4 nm, ±3 nm, ±2 nm, ±1 nm, and so forth. In some cases, the emitter includes a plurality of laser bundles that are each configured to pulse a particular wavelength of EMR with a tolerance threshold not greater than ±5 nm, ±4 nm, ±3 nm, or ±2 nm.

Certain multispectral wavelengths pierce through tissue and enable a medical practitioner to “see through” tissues in the foreground to identify chemical processes, structures, compounds, biological processes, and so forth that are located behind the foreground tissues. The multispectral wavelengths may be specifically selected to identify a specific disease, tissue condition, biological process, chemical process, type of tissue, and so forth that is known to have a certain spectral response.

The variable pulse cycle may include one or more emissions of EMR that are optimized for mapping imaging, which includes, for example, three-dimensional topographical mapping, depth map generation, calculating distances between objects within a scene, calculating dimensions of objects within a scene, determining whether a surgical tool or other object approaches a threshold distance from another object, and so forth. The pulses for laser mapping imaging include EMR formed in a mapping pattern, which may include one or more of vertical hashing, horizontal hashing, a dot array, and so forth.

The controller 104 optimizes the variable pulse cycle to accommodate various imaging and video standards. In most use-cases, the system outputs a video stream comprising at least 30 frames per second (fps). The controller 104 synchronizes operations of the emitter and the image sensor to output data at a sufficient frame rate for visualizing the scene and further for processing the scene with one or more advanced visualization techniques. A user may request a real-time color video stream of the scene and may further request information based on one or more of multispectral imaging, fluorescence imaging, or laser mapping imaging (which may include topographical mapping, calculating dimensions and distances, and so forth). The controller 104 causes the image sensor to separately sense color data frames, multispectral data frames, fluorescence data frames, and mapping data frames based on the variable pulse cycle of the emitter.

In some cases, a user requests more data types than the system can accommodate while maintaining a smooth video frame rate. The system is constrained by the image sensor's ability to accumulate a sufficient amount of electromagnetic energy during each blanking period to output a data frame with sufficient exposure. In some cases, the image sensor outputs data at a rate of 60-120 fps and may specifically output data at a rate of 60 fps. In these cases, for example, the controller 104 may devote 24-30 fps to color visualization and may devote the other frames per second to one or more advanced visualization techniques.

The controller 104 calculates and adjusts the variable pulse cycle of the emitter 102 in real-time based at least in part on the known capabilities of the pixel array 125. The controller 104 may access data stored in memory indicating how long the pixel array 125 must be exposed to a certain waveband of EMR for the pixel array 125 to accumulate a sufficient amount of EMR to output a data frame with sufficient exposure. In most cases, the pixel array 125 is inherently more or less sensitive to different wavebands of EMR. Thus, the pixel array 125 may require a longer or shorter blanking period duration for some wavebands of EMR to ensure that all data frames output by the image sensor 124 comprise sufficient exposure levels.

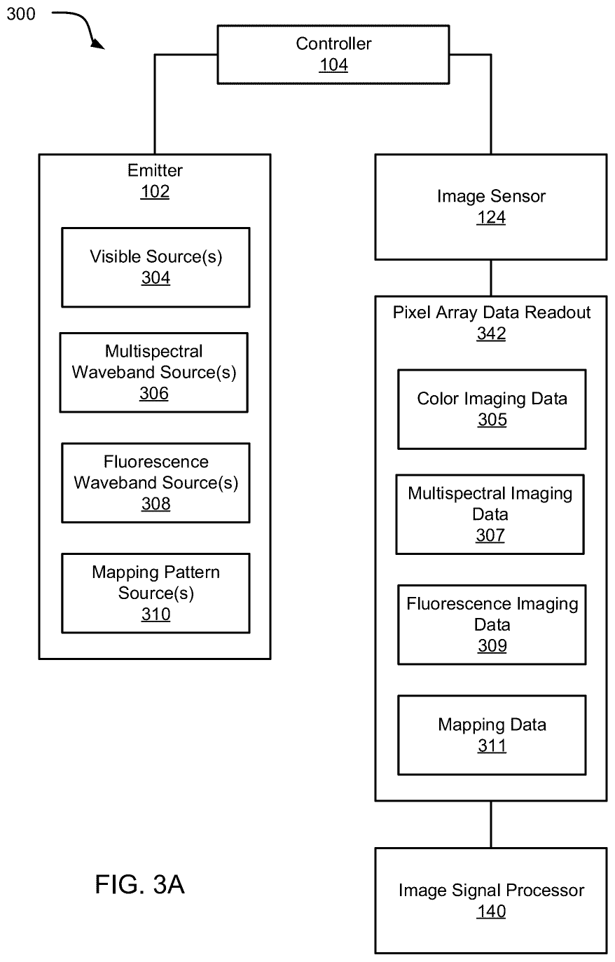

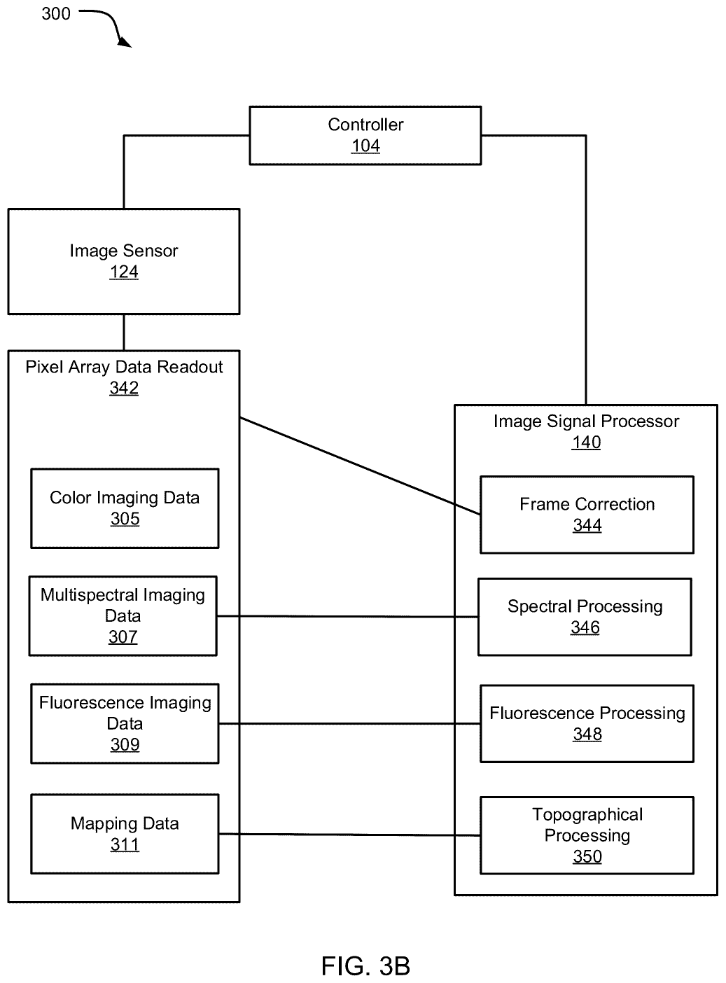

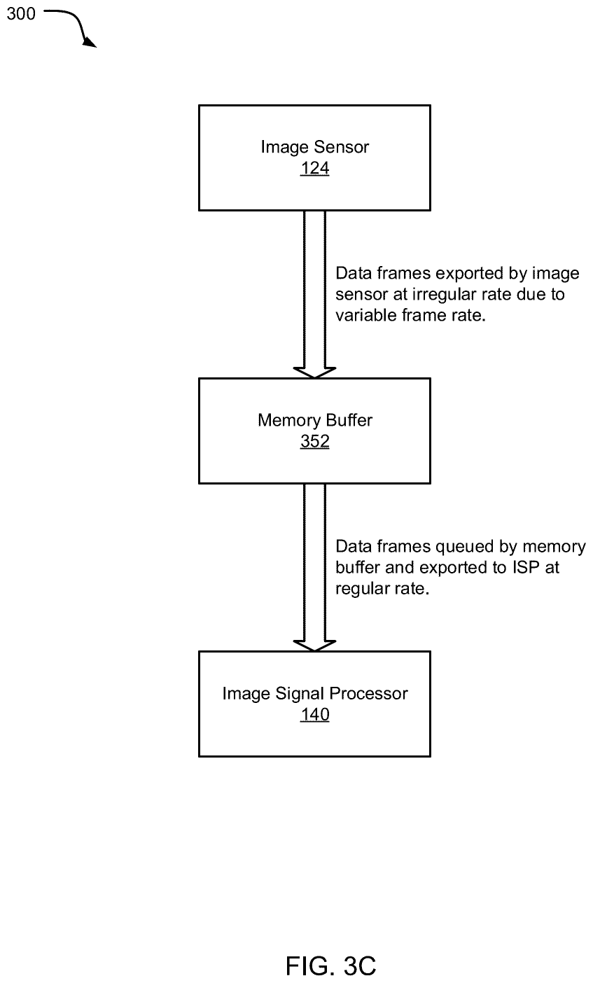

The controller 104 determines the data input requirements for various advanced visualization algorithms (see, e.g., the algorithms 346, 348, 350 first described in FIG. 3B). For example, the controller 104 may determine that certain advanced visualization algorithms do not require a data input at the same regularity as a color video stream output of 30 fps. In these cases, the controller 104 may optimize the variable pulse cycle to include white light pulses at a more frequent rate than pulses for advanced visualization such as multispectral, fluorescence, or laser mapping imaging. Additionally, the controller 104 determines whether certain algorithms may operate with lower resolution data frames that are read out by the image sensor using a pixel binning configuration. In some cases, the controller 104 ensures that all color frames provided to a user are read out in high-resolution (without pixel binning). However, some advanced visualization algorithms (see e.g., 346, 348, 350) may execute with lower resolution data frames.

The system 100 may include a plurality of image sensors 124 that may have different or identical pixel array configurations. For example, one image sensor 124 may include a monochromatic or “color agnostic” pixel array with no filters, another image sensor 124 may include a pixel array with a Bayer pattern CFA, and another image sensor 124 may include a pixel array with a different CFA. The multiple image sensors 124 may be assigned to detect EMR for a certain imaging modality, such as color imaging, multispectral imaging, fluorescence imaging, or laser mapping imaging. Further, each of the image sensors 124 may be configured to simultaneously accumulate EMR and output a data frame, such that all image sensors are capable of sensing data for all imaging modalities.

The controller 104 prioritizes certain advanced visualization techniques based on the user's ultimate goals. In some cases, the controller 104 prioritizes outputting a smooth and high-definition color video stream to the user above other advanced visualization techniques. In other cases, the controller 104 prioritizes one or more advanced visualization techniques over color visualization, and in these cases, the output color video stream may appear choppy to a human eye because the system outputs fewer than 30 fps of color imaging data.

For example, a user may indicate that a fluorescent reagent has been administered to a patient. If the fluorescent reagent is time sensitive, then the controller 104 may ensure that a sufficient ratio of frames is devoted to fluorescence imaging to ensure the user receives adequate fluorescence imaging data while the reagent remains active. In another example, a user requests a notification whenever the user's surgical tool comes within a threshold distance of a certain tissue, such as a blood vessel, nerve fiber, cancer tissue, and so forth. In this example, the controller 104 may prioritize laser mapping visualization to constantly determine the distance between the user's surgical tool and the surrounding structures and may further prioritize multispectral or fluorescence imaging that enables the system to identify the certain tissue. The controller 104 may further prioritize color visualization to ensure the user continues to view a color video stream of the scene.

FIGS. 3A-3C illustrate schematic diagrams of a system 300 for processing data output by an image sensor 124 comprising the pixel array 125. The system 300 includes a controller 104 in communication with each of the emitter 102 and the image sensor 124 comprising the pixel array 125. The emitter 102 includes one or more visible sources 304, multispectral waveband sources 306, fluorescence waveband sources 308, and mapping pattern sources 310 of EMR.

The pixel array data readout 342 of the image sensor 124 includes one or more of color imaging data 305, multispectral imaging data 307, fluorescence imaging data 309, or mapping data 311. The color imaging data 305 may include one or more of a color data frame 205 captured in a time-division system configuration as illustrated in FIGS. 2A-2C or color imaging data captured with a color filter array (CFA) or multispectral filter array (MSFA). The multispectral imaging data 307 may include one or more of a multispectral data frame 207 captured in a time-division system configuration as illustrated in FIGS. 2A-2C or multispectral imaging data captured with a MSFA. The fluorescence imaging data 309 may include one or more of a fluorescence data frame 209 captured in a time-division system configuration as illustrated in FIGS. 2A-2C or fluorescence imaging data captured with a MSFA. The mapping data 311 may include one or more of a mapping data 211 or mapping data calculated with stereoscopic imaging.

When the pixel array 125 is equipped with a MSFA, the color imaging data 305 may be captured simultaneously with one or more of the multispectral imaging data 307, the fluorescence imaging data 309, or the mapping data 311. These data types may be captured simultaneously according to the time-division system configuration discussed in connection with FIGS. 2A-2C, or with a constant illumination system configuration. The type of data extracted from the pixel array data readout 342 will be depending on which EMR sources 134, 138 are cycled on by the emitter 102 during. The emitter 102 may selectively actuate any of visible sources 304, multispectral waveband sources 306, fluorescence waveband sources 308, or mapping pattern sources 310.

When data is captured according to the time-division configuration of FIGS. 2A-2C, the emitter 102 may be instructed to simultaneously cycle on the white EMR source 134 and one or more other EMR sources 138 during a blanking period of the image sensor 124. The one or more other EMR sources 138 may be tuned to emit only EMR within a narrow waveband selected for fluorescence or multispectral visualization. In this configuration, the pixel array 125 with the MSFA may simultaneously capture color visualization data and fluorescence/multispectral visualization data during a single frame period (i.e., a readout period and a blanking period).

In an alternative implementation, the emitter 102 continuously emits one or more EMR sources, including the white EMR source 134 or any of the narrowband EMR sources 138. The emitter 102 may cycle various EMR sources 134, 138 on an off based on user preferences and which datatypes are sought (i.e., color imaging data 305, multispectral imaging data 307, fluorescence imaging data 309, mapping data 311). In this implementation, the pixel array 125 equipped with the MSFA may simultaneously capture color visualization data and fluorescence/multispectral visualization data during each frame period.

As illustrated in FIG. 3B, all data read out by the pixel array may undergo frame correction 344 processing by the image signal processor 140. In various implementations, one or more of the color imaging data 305, the multispectral imaging data 307, the fluorescence imaging data 309, and the mapping data 311 undergoes frame correction 344 processes. The frame correction 344 includes one or more of sensor correction, white balance, color correction, or edge enhancement.

The multispectral imaging data 307 may undergo spectral processing 346 that is executed by the image signal processor 140 and/or another processor that is external to the system 300. The spectral processing 346 may include a machine learning algorithm and may be executed by a neural network configured to process the multispectral imaging data 307 to identify one or more tissue structures within a scene based on whether those tissue structures emitted a spectral response. The spectral processing 346 assesses the pixel integration (accumulation) values for each pixel within the pixel array 125 and may specifically assess the pixel integration values for pixels equipped with an appropriate spectral filter. The pixel integration values will inform the spectral processing 346 algorithm whether a certain pixel likely accumulated a spectral response for a certain tissue, disease, condition, chemical process, biological process, and so forth.

The fluorescence imaging data 309 may undergo fluorescence processing 348 that is executed by the image signal processor 140 and/or another processor that is external to the system 300. The fluorescence processing 348 may include a machine learning algorithm and may be executed by a neural network configured to process to fluorescence imaging data 309 and identify an intensity map wherein a fluorescence relaxation wavelength is detected by the pixel array. The fluorescence processing 348 assesses the pixel integration (accumulation) values for each pixel within the pixel array 125 and may specifically assess the pixel integration values for pixels equipped with an appropriate spectral filter. The pixel integration values will inform the fluorescence processing 348 algorithm whether a certain pixel likely accumulated a fluorescence relaxation emission by a reagent or tissue.

The mapping data 311 may undergo topographical processing 350 that is executed by the image signal processor 140 and/or another processor that is external to the system 300. The topographical processing 350 may include a machine learning algorithm and may be executed by a neural network configured to assess time-of-flight information to calculate a depth map representative of the scene. The topographical processing 350 includes calculating one or more of a three-dimensional topographical map of the scene, a dimension of one or more objects within the scene, a distance between two or more objects within the scene, a distance between a surgical tool and a certain tissue structure within the scene, and so forth.

The topographical processing 350 may additionally or alternatively be based on stereoscopic visualization. The topographical processing 350 may execute stereo imaging triangulation to calculate three-dimensional coordinates of points in a scene using two or more data frames captured from different viewpoints. This is calculated based on the principle of triangulation, which includes measuring relative positions and angles of image sensor 124 viewpoints using the resulting parallax information to determine the depth or distance of objects within a scene. In this case, the topographical processing 350 may include correspondence matching of features in two or more images, disparity estimation, depth calculation, and three-dimensional reconstruction.

FIG. 3C illustrates a schematic diagram of a system 300 and process flow for managing data output at an irregular rate. The image sensor 124 operates according to a sensor cycle that includes blanking periods and readout periods. The image sensor 124 outputs a data frame at the conclusion of each readout period that includes an indication of the amount of EMR the pixel array accumulated during the preceding accumulation period or blanking period.

Each frame period in the sensor cycle is adjustable on a frame-by-frame basis to optimize the output of the image sensor and compensate for the pixel array 125 having varying degrees of sensitivity to different wavebands of EMR. The duration of each blanking period may be shortened or lengthened to customize the amount of EMR the pixel array 125 can accumulate. Additionally, the duration of each readout period may be shortened or lengthened by implementing a pixel binning configuration or causing the image sensor to read out each pixel within the pixel array 125. Thus, the image sensor 124 may output data frames at an irregular rate due to the sensor cycle comprising a variable frame rate. The system 300 includes a memory buffer 352 that receives data frames from the image sensor 124. The memory buffer 352 stores the data frames and then outputs each data frame to the image signal processor 140 at a regular rate. This enables the image signal processor 140 to process each data frame in sequence at a regular rate.

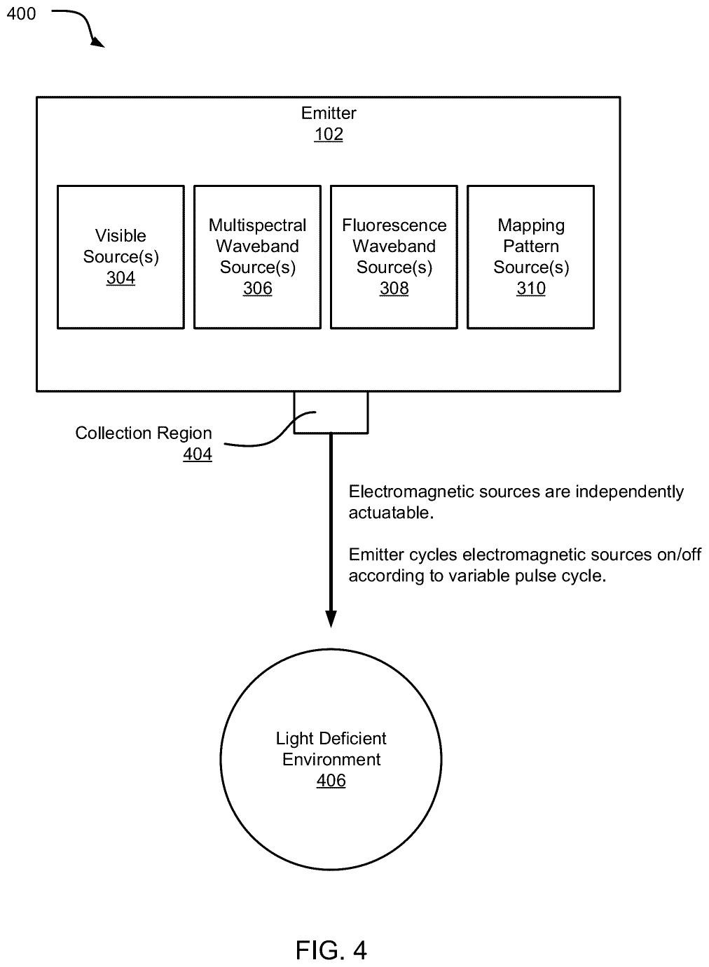

FIG. 4 is a schematic diagram of an illumination system 400 for illuminating a light deficient environment 406 such as an interior of a body cavity. In most cases, the emitter 102 is the only source of illumination within the light deficient environment 406 such that the pixel array of the image sensor does not detect any ambient light sources. The emitter 102 includes a plurality of separate and independently actuatable sources of EMR, which may include visible source(s) 304, multispectral waveband source(s) 306, fluorescence waveband source(s) 308, and mapping pattern source(s) 310. The emitter may cycle a selection of the sources on and off to pulse according to the variable pulse cycle received from the controller 104. Each of the EMR sources feeds into a collection region 404 of the emitter 102. The collection region 404 may then feed into a waveguide (see e.g., 130 in FIG. 1A) that transmits the pulsed EMR to a distal end of an endoscope within the light deficient environment 406.

The variable pulsing cycle is customizable and adjustable in real-time based on user input. The emitter 102 may instruct the individual EMR sources to pulse in any order. Additionally, the emitter 102 may adjust one or more of a duration or an intensity of each pulse of EMR. The variable pulse cycle may be optimized to sufficiently illuminate the light deficient environment 406 such that the resultant data frames read out by the pixel array 125 are within a desired exposure range (i.e., the frames are neither underexposed nor overexposed). The desired exposure range may be determined based on user input, requirements of the image signal processor 140, and/or requirements of a certain image processing algorithm (see 344, 346, 348, and 350 in FIG. 3B). The sufficient illumination of the light deficient environment 406 is dependent on the energy output of the individual EMR sources and is further dependent on the efficiency of the pixel array 125 for sensing different wavebands of EMR.

FIG. 5 is a schematic diagram of an endoscope assembly 500 for providing visualization of a light-deficient environment. The endoscope assembly 500 includes at least an endoscope, handpiece, and cable. The endoscope assembly 500 is configured to interface with one or more of the emitter 102 or controller 104 described herein. The endoscope assembly 500 may include any of the components described in connection with the system 100 first described in connection with FIGS. 1A-1C and may specifically include components of the endoscope 108, handpiece 154, waveguides, and data transmission cables.

The endoscope assembly 500 is configured to interface with the emitter 102 and the controller 104. The emitter 102 and the controller 104 may communicate with the endoscope 108 by way of an integrated cable 502a, 502b (may collectively be referred to as integrated cable 502 as described herein). In some implementations, the integrated cable 502 includes a rigid integrated cable 502a directly attached to the handpiece 154, and a flexible integrated cable 502b attached to the rigid integrated cable 502a. The flexible integrated cable 502b terminates at an integrated connector 504. The integrated connector 504 is configured to interface with a corresponding electronic and optical connector associated with the emitter 102 and controller 104. The integrated cable 502 may include one or more of the waveguide 130, the waveguide 131, or the data transmission pipeline 146 described in connection with FIGS. 1A-1C and may additionally include other cables as needed.

The endoscope 108 includes a handpiece 154 that is releasably or permanently attached to the insertion tube 112. The endoscope includes the visualization system 106 disposed at a distal end of the insertion tube 112. The visualization system 106 may include one or more of the components of the visualization system 106 first described in connection with FIGS. 1A-1C. The visualization system 106 specifically includes components of an optical-electronic payload (OEP), including, for example, lenses, filters, prisms, mirrors, image sensors, pixel arrays, processors, field programmable gate arrays, printed circuit boards, microcontrollers, controllers, and so forth. The visualization system 106 additionally includes illumination components, which may include fiber optic bundles configured to transmit EMR from an emitter.

The endoscope 108 is a key component of a system for minimally invasive surgery in both robotic and manual surgical implementations. Considering the cost and manufacturing complexity of the endoscope 108, it can be important to ensure the endoscope 108 is suitable for reprocessing, which includes cleaning, disinfecting, and sterilization. In many cases, it is preferable to perform sterilization with autoclave techniques, due to the low cost, low labor requirement, and high efficiency of autoclave sterilization.

Autoclaving includes high-pressure steam sterilization, and it is known to be an effective and economic means for equipment sterilization. The basic principles of autoclave sterilization include exposing the device to direct steam contact at the required temperature and pressure for a specified duration of time. Thus, there are at least three main parameters of steam sterilization, including pressure, temperature, and time.

Autoclave sterilization cycles may be divided into three distinct phases, including a conditioning phase, an exposure phase, and a drying phase. During the condition phase, air is removed from the autoclave load, and the devices within the autoclave load (e.g., an endoscope 108 like the one illustrated in FIG. 5) are heated to a desired temperature for sterilization. The conditioning phase is important because if any air remains within the autoclave load, the air may prevent the sterilant from coming into contact with the devices undergoing sterilization, and this leads to sterilization failure. During the exposure phase, the devices undergoing sterilization are held at a specific temperature for a time known to provide effective sterilization. During the drying phase, steam is removed from the autoclave load, and the devices undergoing sterilization are dried to prevent recontamination of instruments through wicking of microorganisms through a wet wrap.

Typical conditions for the autoclave sterilization cycle are described in Table 1.

TABLE 1

| | | | Reference Value | |
|---|---|---|---|---|
| | Parameter | Specification | (Imperial) | |
| 1 | Vacuum | −880 mbar (=133 mbar abs.) | −26 | inHg |
| 2 | Pressure (Steam) | 2206 mbar (=3219 mbar abs.) | 32 | psig |
| 3 | Temperature | 134° Celsius | 273 | F. |
| 4 | Sterilization | 4 minutes (United States) or | 18 | minutes |

| | |
|---|---|
| Time | 18 minutes (European Union) |

As shown in Table 1, the sterilization process exposes the devices undergoing sterilization (like the endoscope 108 described herein) to harsh conditions. These harsh conditions may be repeated for multiple reprocessing cycles over the lifetime of the endoscope (typically more than 200 cycles). Therefore, design and manufacturing of the endoscope 108 shall meet these reprocessing conditions to ensure the endoscope 108 is suitable for autoclave sterilization.

The systems described herein are designed such that a complete endoscope assembly, including at least the endoscope 108, the integrated cable 502, and the integrated connector 504, can undergo an autoclave sterilization cycle as a single unit. For the endoscope assembly to sustain autoclave sterilization, the optical and electronic components inside at least the endoscope 108, the integrated cable 502, and the integrated connector 504 shall be protected from moisture ingress. The endoscope assembly is hermetically sealed to ensure the sensitive components are maintained under vacuum and pressure conditions as required during the autoclave sterilization cycle.

As described herein, various junctions and components of the endoscope assembly are sealed utilizing laser welding (i.e., joining metal components via laser induced melting of a base material) or laser brazing (i.e., joining metal and non-metal components via laser induced milting of a filter material). These sealing processes create watertight and airtight barriers that endure multiple repetitive high-temperature, and high-pressure (both positive and negative) cycles as occur during autoclave sterilization processes. Additionally, the endoscope assembly may undergo additional sealing processes, including epoxy gluing individual optical fibers into a combined bundle. The epoxied optical fiber optic bundle is less resistive to water molecules wicking along the microscopic cavities of the optical fibers, which may develop after repetitive sterilization cycles using harsh solvents.

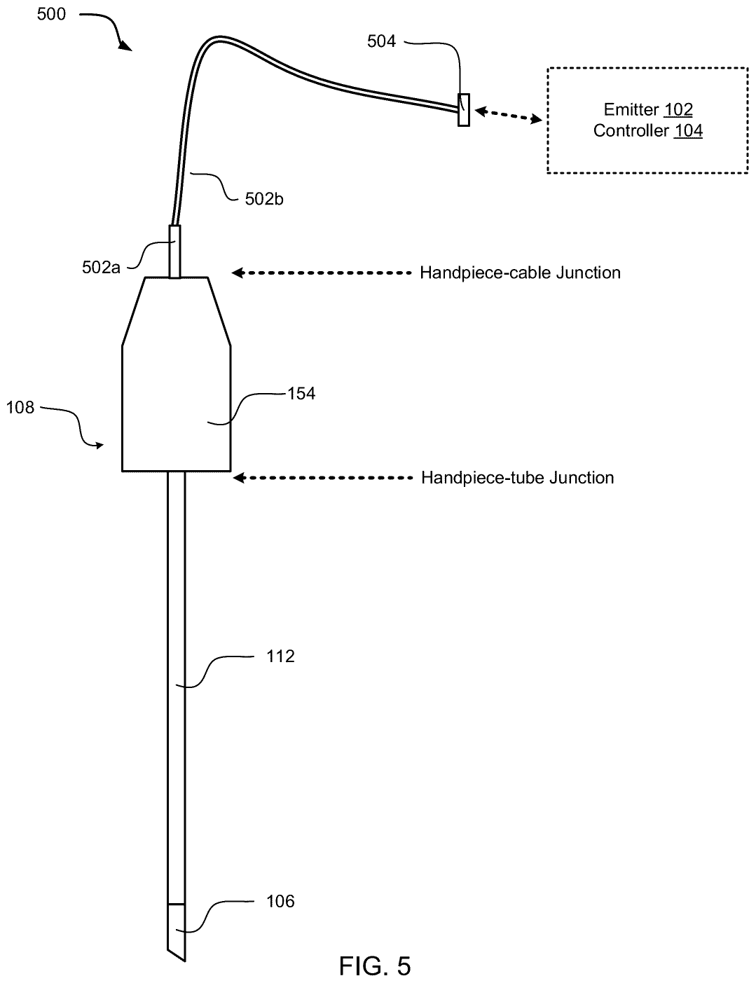

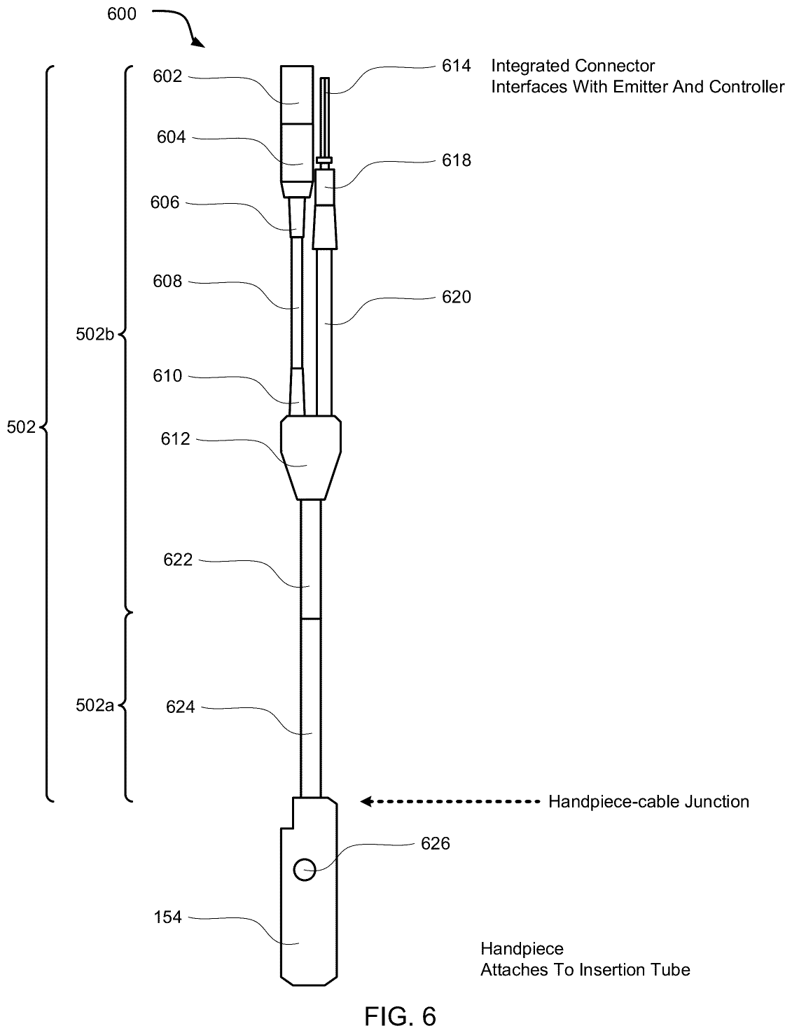

FIG. 6 is a schematic illustration of an example optical-electronic pipeline 600. The optical-electronic pipeline 600 includes the integrated cable 502 and additionally includes the handpiece 154 of the endoscope 108. The optical-electronic pipeline 600 provides optical communication between the handpiece 154 of the endoscope 108 and the emitter 102. The optical-electronic pipeline 600 additionally provides electronic or data transmission communication between the handpiece 154 of the endoscope 108 and the controller 104.

The integrated cable 502 may ultimately terminate at an integrated connector (see 504) as shown in FIG. 5, wherein both optical transmission and data transmission are combined into a single plug. In alternative implementations, the integrated cable 502 may split into a separate optical connector and data transmission connector. This may be desirable if the emitter 102 and the controller 104 are encased in different housings, or if the emitter 102 and the controller 104 have separate interfaces for communicating illumination and data.

The integrated cable 502 includes optical components for transmitting electromagnetic radiation, and additionally includes electronic components for transmitting data and/or power. The data transmission components terminate at an end cap 602 and end plug 604. The end plug 604 is configured to interface with a corresponding data transmission receptacle disposed within a housing for the controller 104. The data transmission components additionally include a kink protection 606 that bridges in between the end plug 604 and a flexible data cable 608. The data transmission components further include a kink protection 610 that bridges in between the flexible data cable 608 and a splitter 612. The splitter 612 may specifically include a Y-splitter.

The optical components terminate at a fiber connector 614 that is configured to interface with a corresponding optical receptacle disposed within a housing for the emitter 102. The optical components further include a fiber handle 618 that provides protection for delicate fiber optic bundle(s) terminating at the fiber connector 614. The optical components include a fiber optic hose 620 that encases one or more fiber optic bundles. The fiber optic hose 620 terminates at the splitter 612.

The integrated cable 502 includes a flexible cable housing 622 that joins with a rigid cable housing 624. The rigid integrated cable 502a includes the rigid cable housing 624 to interface with the handpiece 154 of the endoscope 108. The rigid cable housing 624 and the handpiece 154 may each be constructed of a metal material. The rigid cable housing 624 may be welded or laser brazed to the metal housing of the handpiece 154 to ensure a hermetic seal.

The handpiece 154 includes an actuator 626 disposed within a housing of the handpiece 154. The actuator 626 may specifically include a button that hermetically seals when the endoscope 108 undergoes an autoclave sterilization cycle.

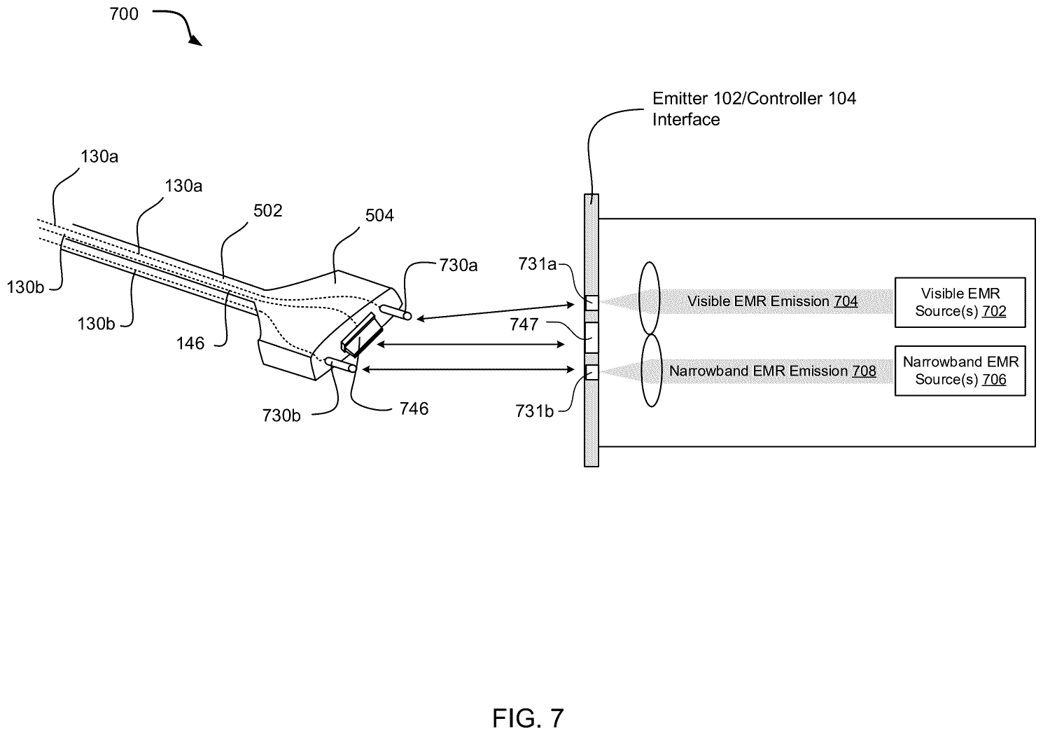

FIG. 7 is a schematic illustration of a system 700 for facilitating optical and electronic communication between an endoscope (see 108) and an emitter 102 and controller 104. The system 700 includes the integrated cable 502 and integrated connector 504 and illustrates wherein the integrated connector 504 interfaces with corresponding ports disposed within an emitter 102/controller 104 interface.

In some implementations, the integrated connector 504 includes a plurality of fiber optic ferrules, wherein each of the plurality of fiber optic ferrules is in optical communication with a different fiber optic bundle, and wherein each fiber optic bundle is dedicated to transmitting a distinct type of EMR. In alternative implementations, the integrated connector 504 includes a singular optical ferrule that is in optical communication with a fiber optic bundle that transmits varying types of EMR.

In the exemplary implementation illustrated in FIG. 7, the integrated connector 504 includes a first fiber optic ferrule 730a and a second fiber optic ferrule 730b. The first fiber optic ferrule 730a is configured to be coupled to a first waveguide contact 731a that is integrated into a housing of the emitter 102. When the first fiber optic ferrule 730a is coupled to the first waveguide contact 731a, the first waveguide 130a may receive visible EMR emission 704 that is pulsed by one or more visible EMR sources 702 of the emitter 102. The first waveguide 130a may then transmit this visible EMR emission 704 to a distal end of an endoscope (see 108). The second fiber optic ferrule 730b is configured to be coupled to a second waveguide contact 731b that is integrated into a housing of the emitter 102. When the second fiber optic ferrule 730b is coupled to the second waveguide contact 731b, the second waveguide 130b may receive a narrowband EMR emission 708 that is pulsed by one or more narrowband EMR sources 706 of the emitter 102. The second waveguide 130b may then transmit the narrowband EMR emission 708 to a distal end of an endoscope (see 108).

The integrated connector 504 further includes a data connector 746 that is configured to interface with a corresponding data port 747 of the emitter 102/controller 104 interface. This enables bidirectional data communication between the controller 104 and one or more processing components of an endoscope (see 108). This may specifically enable bidirectional data communication between the controller 104 and one or more of the image sensor (see 124) or microcontroller (see 122). The bidirectional data communication may be carried out on the data transmission pipeline 146.

The configuration illustrated in FIG. 7 may enable increased luminous efficiency when compared with an alternative embodiment wherein EMR from the visible EMR sources 702 and the narrowband EMR sources 706 are combined within the emitter 102 itself. If the visible EMR sources 702 and the narrowband EMR sources 706 were combined within the emitter 102, then the system 100 may experience light loss at each junction.

FIG. 8 is a schematic illustration of a perspective view of the integrated connector 504. As described herein, the integrated connector 504 may have varying numbers of data transmission ports and optical ports, depending on the implementation. In the exemplary implementation illustrated in FIG. 8, the integrated connector 504 includes a single data connector 802 and a single fiber optic ferrule 804. The fiber optic ferrule 804 includes a first fiber optic coupler 806 and a second fiber optic coupler 808. The first and second fiber optic couplers 806, 808 may receive several types of EMR, as described in connection with FIG. 7.

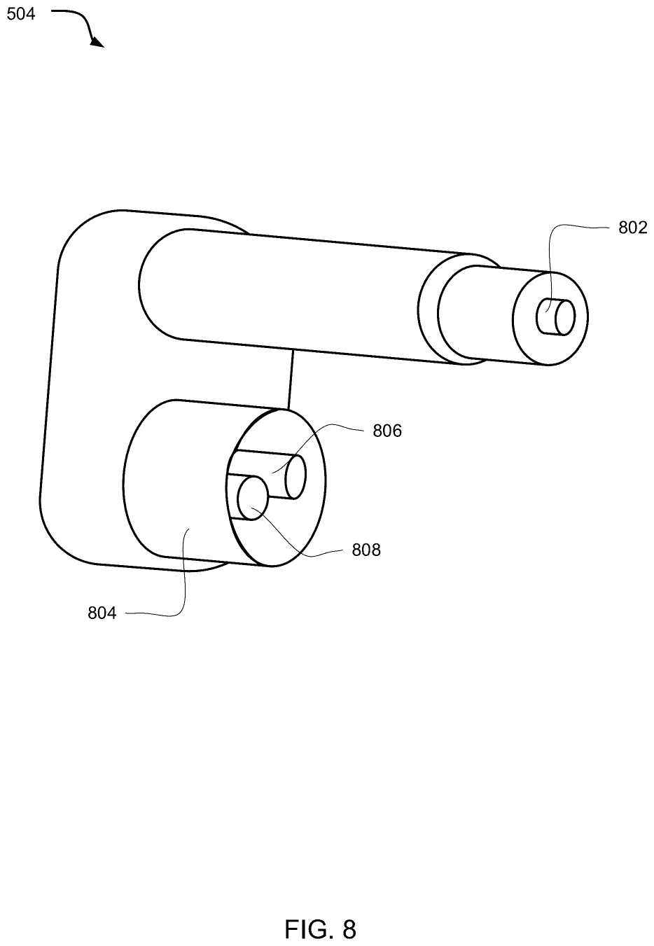

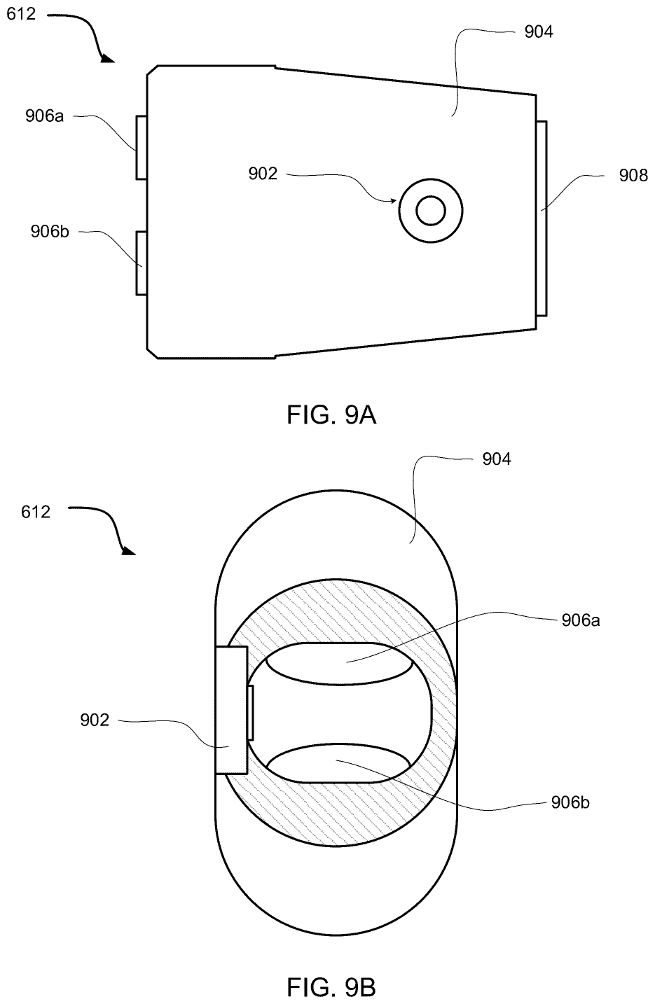

FIGS. 9A-9C illustrate various views of the splitter 612 of the optical-electronic pipeline (see 600 first described in connection with FIG. 6). FIG. 9A is a schematic illustration of a side view of the splitter 612. FIG. 9B is a schematic illustration of a cross-sectional view of the splitter 612. FIG. 9C illustrates a cross-sectional perspective view of the splitter 612. The splitter 612 is configured to receive a singular cable exiting the handpiece (see 154), and then split that singular cable into separate optical and data transmission components.

In some implementations, the splitter 612 includes a microvalve 902 disposed within an external wall 904 of the splitter 612. The microvalve 902 is configured to release pressure within an internal cavity of the splitter 612, and further within the integrated cable (see 502) as a whole. The sheath of the integrated cable 502 may experience significant pressure when the endoscope system (including at least the endoscope 108, handpiece 154, integrated cable 502, and integrated connector 504) undergoes an autoclave sterilization cycle. The sheath of the integrated cable 502 may be at risk of bursting when the integrated cable 502 undergoes a vacuum phase or dry phase of the autoclave sterilization cycle. This is due to differential pressure building inside the tightly sealed integrated cable 502. The microvalve 902 is designed and calibrated to release pressure during the negative pressure autoclave cycles (including the vacuum phase and dry phase). The microvalve 902 is designed to exclusively open when inner pressure inside the integrated cable 502 exceeds outer pressure outside the integrated cable 502. Otherwise, the microvalve 902 is tightly closed by the outer pressure (may include atmospheric pressure or positive pressure applied by an autoclave sterilization system) to prevent moisture ingress into an interior of the integrated cable 502.

The pressure release microvalve 902 may be integrated into the external wall 904 of the splitter 612. As described herein, the splitter 612 may be a component of the integrated cable 502. The splitter 612 may specifically be utilized to separate a singular cable portion of the integrated cable 502 (i.e., wherein optical components and data transmission components are encased in a single sheath), and a separated cables portion of the integrated cable 502 (i.e., wherein optical components and data transmission components are encased in separate sheaths).

As shown in FIGS. 9A-9C, the microvalve 902 is seated into the external wall 904 of the splitter 612. The splitter 612 includes a first input hole 906a and a second input hole 906b disposed at one side of the external wall 904. The splitter 612 additionally includes an output hole 908 disposed at an opposite side of the external wall 904. The microvalve 902 is located in between the output hole 908 and the first/second input holes 906a, 906b. In some cases, the microvalve 902 is located nearer to the output hole 908 than the first/second input holes 906a, 906b.

As shown in FIG. 9C, the microvalve 902 may be seated within the external wall 904 at an optimized depth, which is referred to as the seat thickness 910. The seat thickness 910 and the thickness of the external wall 904 may be optimized to ensure the integrated cable and splitter 612 are capable of undergoing an autoclave sterilization cycle, and further to ensure the microvalve 902 only opens in response to a threshold amount of negative pressure.

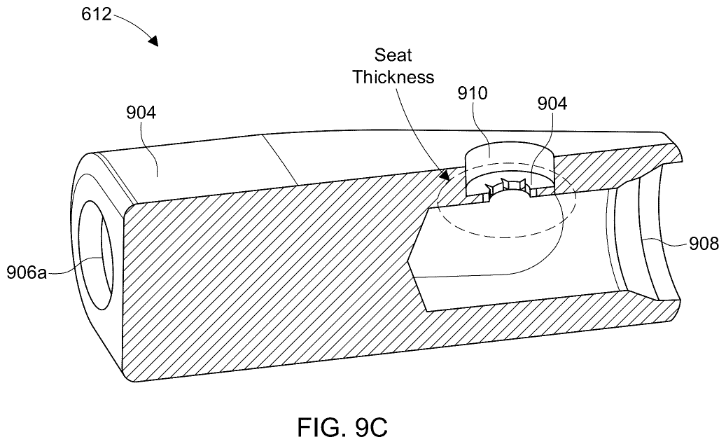

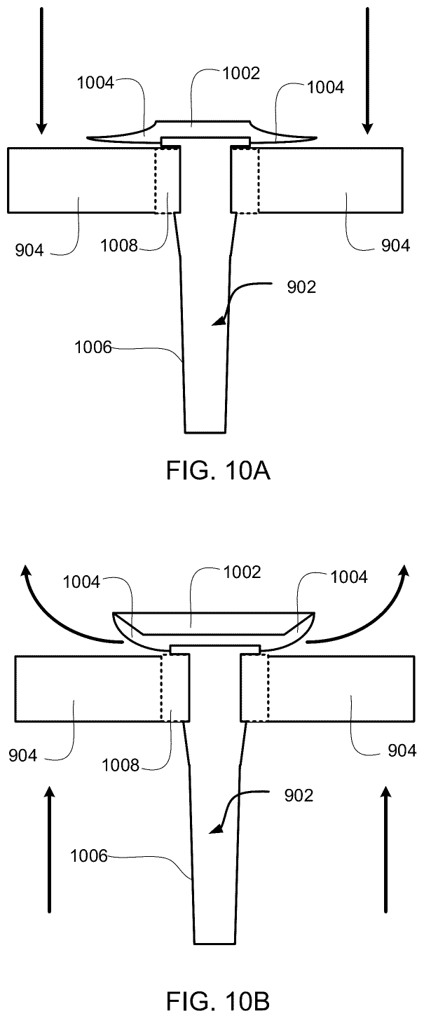

FIGS. 10A and 10B are schematic illustrations of the microvalve 902 for releasing pressure within an integrated cable (see 502) of an endoscope (see 108). FIG. 10A illustrates wherein the microvalve 902 is in a closed position, and FIG. 10B illustrates wherein the microvalve 902 is in the open position. The microvalve 902 may be embedded within a splitter (see 612) of an integrated cable (see 502) of the endoscope. The microvalve 902 may alternatively be embedded into other components of the endoscope, including within other components of an integrated cable.

The microvalve 902 is configured to burst open and release pressure during one or more of a vacuum phase or a dry phase of an autoclave sterilization cycle. Thus, in some cases, the microvalve 902 opens in response to the internal pressure of an autoclave chamber being negative due to decompression. When the autoclave chamber has negative pressure, then an internal pressure within the integrated cable will exceed an external pressure outside the integrated cable. The microvalve 902 automatically opens to normalize the differential in pressure between the autoclave chamber and the interior of the integrated cable.

The endoscope (see 108) and associated integrated cable (see 502) are configured to undergo an autoclave sterilization cycle as a single unit. The autoclave sterilization cycle includes one or more sequences when pressure within the autoclave chamber is reduced. When the pressure within the autoclave chamber is reduced, the internal pressure within the integrated cable will increase. This pressure differential may potentially cause a sheath of the integrated cable to burst during the autoclave sterilization cycle. The microvalve 902 is designed to relive internal pressure within the integrated cable by opening like an umbrella when then internal pressure of the integrated cable exceeds the exterior pressure outside the integrated cable. The microvalve 902 thereby releases the high internal pressure within the integrated cable and protects the integrated cable from bursting during the autoclave sterilization cycle.

The microvalve 902 provides a compensation mechanism for an outer sheath of the integrated cable to maintain its integrity when the inner pressure of the integrated cable builds during an autoclave sterilization cycle. Because both ends of the integrated cable are tightly sealed (i.e., at the cable-handpiece junction and at the cable-connector junction), air cannot quickly release through micropores within the sheath of the integrated cable. The microvalve 902 selectively opens only when the inner pressure on the microvalve 902 exceeds the outer pressure on the microvalve 902. This enables air to quickly release from within the integrated cable without applying excessive pressure on the sheath of the integrated cable.

The diameter of the microvalve 902, the seat thickness (see 910), and the number and diameter of holes within the splitter define the opening pressure for the microvalve 902. These specifications may be optimized and calibrated to ensure the microvalve 902 automatically and exclusively opens when the internal pressure within the integrated cable exceeds the external pressure outside the integrated cable by a threshold amount.

As shown in FIGS. 10A and 10B, the microvalve 902 may be seated within the external wall 904 of a component of the integrated cable. As described herein, the microvalve 902 may specifically be seated within the external wall 904 of a splitter (see 612). The microvalve 902 includes a head 1002 that comprises an umbrella 1004. The microvalve 902 further includes a shaft 1006 attached to the head 1002, wherein the shaft 1006 is disposed through a hole 1008 formed in the external wall 904.

As shown in FIG. 10A, the microvalve 902 is in the closed position, with the umbrella 1004 resting against the external surface of the external wall 904, when positive pressure or atmospheric pressure presses against the microvalve 902 from outside. As shown in FIG. 10B, when the internal pressure exceeds the exterior pressure, and thus, the internal pressure presses against the external wall 904, then the umbrella 1004 of the microvalve 902 will turn upward. This enables air to release through the hole 1008 formed in the external wall 904.

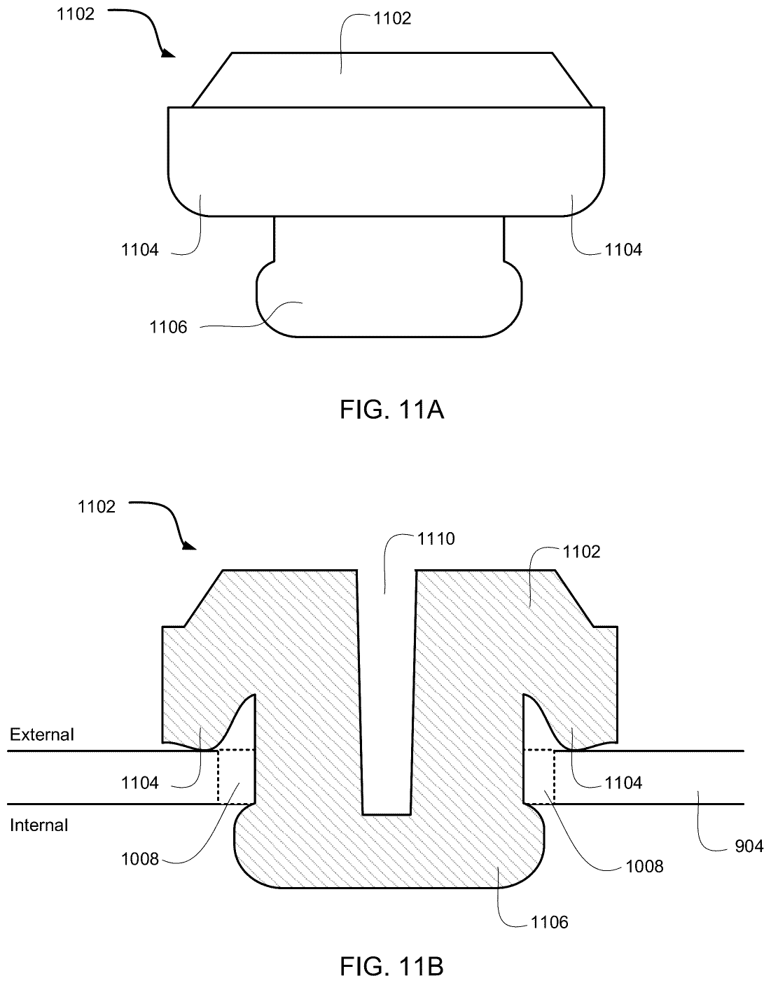

FIGS. 11A and 11B are schematic illustrations of a microvalve 1102 for releasing pressure within an endoscope (see 108). The microvalve 1102 may be disposed within the external wall of the splitter as shown in FIGS. 9A-9C. FIG. 11A is a schematic straight-on side view of the microvalve 1102, and FIG. 11B is a schematic cross-sectional side view of the microvalve 1102.

The microvalve 1102 includes a head 1102 that comprises an umbrella 1104. The microvalve 1102 additionally includes a shaft 1106 attached to the head 1102. As shown in FIG. 11B, the shaft 1106 of the microvalve is disposed through the hole 1008 formed in the external wall 904 of a component of the integrated cable. The microvalve 1102 additionally includes a blind hole 1110 cut into the head 1102. The blind hole 1110 serves as a flex point and enables the umbrella 1104 to flex inward and upward in response to the internal pressure exceeding the external pressure. When the internal pressure exceeds the external pressure by a threshold amount, the head 1102 and umbrella 1104 will flex inward, and this flex is permitted due to the blind hole 1110. Additionally, the umbrella 1104 flexes upward and away from an external surface of the external wall 904. This enables air to escape through the hole 1008 formed in the external wall 904.

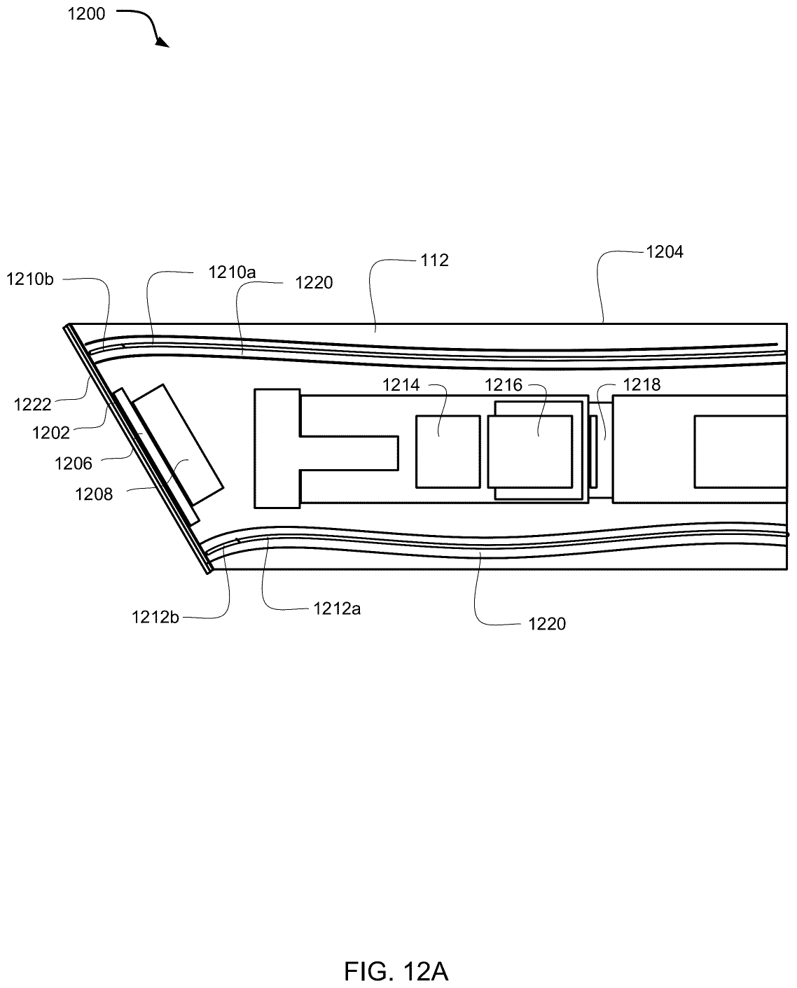

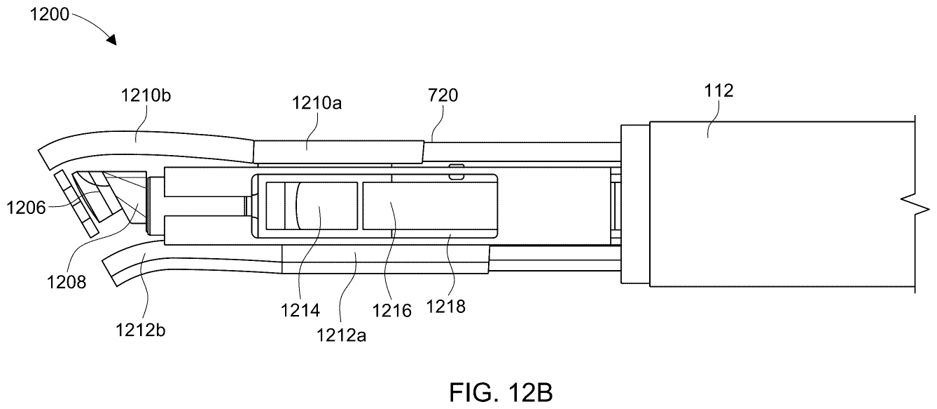

FIGS. 12A and 12B are schematic views of a visualization system 1200. FIG. 12A is a schematic cross-sectional side view of the visualization system 1200 of an endoscope, wherein the visualization system 1200 may be disposed at a distal end of an insertion tube 112 of the endoscope. FIG. 12B is a cutaway illustration of components of the visualization system 1200, wherein at least a portion of the insertion tube 112 is removed, a window is removed, and a cover plate is removed. The visualization system 1200 illustrated may be implemented in any of the systems, methods, and devices described herein, and including in the endoscope 108 first described in connection with FIGS. 1A-1C. As described herein, the visualization system 1200 may be disposed at a distal end of an endoscopic insertion tube 112 such that the endoscope includes a chip-on-tip architecture.

The visualization system 1200 includes illumination components and an optical-electronic payload (OEP) disposed within the same physical space. The visualization system 1200 includes numerous components to ensure airtight and watertight seals to prevent the ingress of moisture during an autoclave sterilization cycle. The systems, methods, and devices for sealing described herein enable the illumination components (e.g., optical fiber bundles for transmitting EMR) and the optical-electronic payload components (e.g., lenses, prisms, filters, mirrors, image sensors, pixel arrays, printed circuit boards, processors, microcontrollers, controllers, heat sinks, and so forth) to be disposed within the same physical space without constructing separate mechanical housings. This minimizes the total radial dimension of all combined components and allows the visualization system 1200 to be disposed within a small insertion tube 112.

The visualization system 1200 is designed to prevent the ingress of moisture that could damage the delicate optical and electronic components disposed within the insertion tube. The visualization system 1200 is thus designed to withstand numerous autoclaving sterilization cycles, which introduce high-pressure and high-temperature steam. The visualization system 1200 includes a cover plate 1222 and a window 1202 disposed across an entirety of the distal end of the insertion tube. The window 1202 may be constructed of an optical-grade components, such as an optical-grade glass or sapphire material. The window 1202 is hermetically sealed to the cover plate 1222 and prevents the ingress of moisture into the interior space of the insertion tube 112.

The visualization system 1200 includes one or more optical elements 1206, 1208. The optical elements 1206, 1208 may include, for example, lenses, prisms, filters, mirrors, and so forth. The optical elements 1206, 1208 are selected to optimize image quality and direct the greatest amount of light toward the image sensors (see, e.g., 124 first described in connection with FIGS. 1A-1C). The optical elements 1206, 1208 are leveraged to focus light on to a pixel array of an image sensor that is disposed within the insertion tube 112.

The visualization system 1200 additionally includes illumination components, which may specifically include one or more fiber optic cables. The visualization system 1200 illustrated in FIGS. 12A and 12B includes a first fiber optic cable 1210a, 1210b and additionally includes a second fiber optic cable 1212a, 1212b. The fiber optic cables 1210a-1210b, 1212a-1212b may be dedicated to transmitting diverse types of EMR from an emitter to a distal end of the insertion tube 112. For example, the first fiber optic cable 1210a, 1210b may be dedicated to transmitting white EMR to the distal end of the insertion tube 112. The second fiber optic cable 1212a, 1212b may be dedicated to transmitting wavelengths of EMR selected for fluorescence excitation or spectral visualization. In some implementations, the fiber optic cables 1210a-1210b, 1212a-1212b are bifurcated as shown in FIGS. 12A and 12B. Thus, the first fiber optic cable 1210a, 1210b may include a proximal portion (see 1210a) and a distal portion (see 1210b). Additionally, the second fiber optic cable 1212a, 1212b may include a proximal portion (see 1212a) and a distal portion (see 1212b). The proximal and distal portions of the fiber optic cables may butt against each other to ensure efficient transmission of EMR from the proximal portion to the distal portion.

The visualization system 1200 includes a ferrule enclosure 1220 disposed around each of the fiber optic cables 1210a-1210b, 1212a-1212b. The ferrule enclosure 1220 may be constructed of metal and may extend a length of the visualization system 1200. The ferrule enclosure 1220 serves to protect the fiber optic cables 1210a-1210b, 1212a-1212b from moisture damage, and further serves to protect the optical and electronic components disposed within the insertion tube 112.

The optical-electronic payload of the visualization system 1200 includes one or more image sensors 1214. As shown in FIG. 1B, a plane of the image sensor 1214 may be oriented substantially parallel to a longitudinal axis of the insertion tube 112. This may be contrary to conventional image sensor placement, wherein the image sensor would be pointed directly at a scene and thus, the plane of the image sensor would be oriented substantially perpendicular to the longitudinal axis of the insertion tube 112. However, the conventional image sensor placement is difficult to achieve within the extreme size constraints of the insertion tube 112, and this is particularly true when utilizing two or more image sensors for stereo visualization. The visualization system 1200 may thereby include two or more image sensors 1214 that are oriented substantially parallel to one another, and substantially parallel to a longitudinal axis of the insertion tube 112. The visualization system 1200 may include additional optical components, such as lenses, prisms, and mirrors for reflecting and transmitting light on to the pixel arrays of the image sensors 1214.

The visualization system 1200 includes a thermal heat sink 1216 for collecting and dissipating heat output by the electronic components disposed within the insertion tube 112. The visualization system 1200 includes one or more printed circuit boards 1218 (CPBs) for facilitating electronic communication to and from the image sensors 1214 and other electronic components.

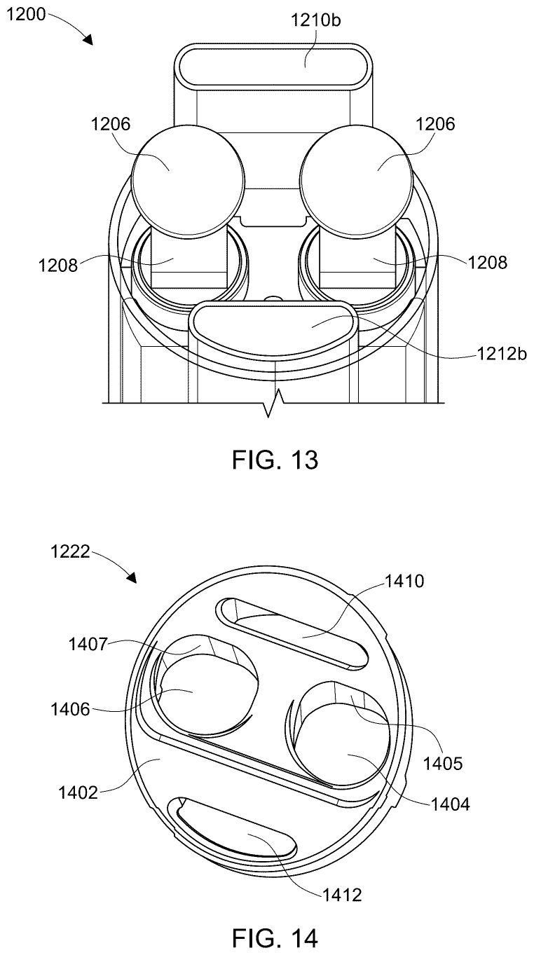

FIG. 13 is a cross-sectional perspective view of a portion of the distal end of the visualization system 1200. In FIG. 13, the window (see 1202) and cover plate (see 1222) are removed. As shown in FIG. 13, The visualization system 1200 may include a stacked lens 1206 and prism 1208 pair for each of two image sensor (see 124) disposed within the insertion tube 112.

The visualization system 1200 is designed to be compatible with autoclave sterilization procedures even when the optical and electrical components are disposed within a small insertion tube 112, such as an insertion tube 112 with a sub-9 mm diameter. The visualization system 1200 includes a hermetically sealed protective sapphire window over each optical channel to create a separation barrier between the interior of the insertion tube 112 and the exterior of the insertion tube 112. The visualization system 1200 may additionally include a metal ferrule enclosure for optical fibers that extends the length of the visualization system 1200. The visualization system 1200 may utilize a glass-potted data cable connector with an embedded optical fiber bundle as a hermetical seal to separate an interior of the insertion tube 112 from an exterior of the insertion tube 112. This glass-to-metal connector may be disposed at the interface wherein an integrated cable enters the handpiece (see 154) of the endoscope (see 108).

FIG. 14 is a perspective view of a cover plate 1222 of the visualization system 1200. The cover plate 1222 is utilized to protect the delicate optical and electronic components of the visualization system 1200. The cover plate 1222 includes a distal surface 1402 that will be sealed to the window (see 1202) of the visualization system 1200.

The cover plate 1222 includes varying numbers of holes depending on the implementation. Additionally, the sizes and geometries of the holes will vary depending on the implementation. The cover plate 1222 illustrated in FIG. 14 corresponds with the implementation illustrated in FIG. 13, such that the cover plate 1222 includes a first fiber optic hole 1410, a second fiber optic hole 1412, a first visualization hole 1404, and a second visualization hole 1406. The first fiber optic hole 1410 corresponds with the first fiber optic cable 1210a, 1210b. The second fiber optic hole 1412 corresponds with the second fiber optic cable 1212a, 1212b. The first visualization hole 1404 corresponds with an optical assembly for a first image sensor (see 124). The second visualization hole 1406 corresponds with an optical assembly for a second image sensor (see 124).

The cover plate 1222 is constructed of a metal material. The cover plate 1222 is welded or laser brazed to other components of the visualization system 1200 to ensure a hermetic seal that prevents that ingress of moisture during an autoclave sterilization cycle. The boundaries of the first fiber optic hole 1410 are welded or laser brazed to the ferrule enclosure 1220 for the first fiber optic cable 1210a, 1210b. The boundaries of the second fiber optic hold 1412 are welded or laser brazed to the ferrule enclosure 1220 for the second fiber optic cable 1212a, 1212b. The welding or laser brazing is utilized to ensure a hermetic seal and to thus prevent moisture from reaching the fiber optic bundles and then wicking up the fiber optic bundles toward delicate electronic components.

The distal surface 1402 of the cover plate 1222 is laser brazed to the window (see 1202) to further ensure a hermetic seal. This further prevents the ingress of moisture into an interior space of the insertion tube (see 112) during an autoclave sterilization cycle.

The visualization system 1200 is designed to prevent moisture ingress that could damage the delicate optical and electronic components disposed at a distal end of an insertion tube. As shown in FIG. 12A, each optical channel is encased by a metal ferrule enclosure 1220. The ferrule enclosure 1220 is laser welded or laser brazed to a fiber optic hole (see 1410, 1412) of the cover plate 1222. In some implementations, the first visualization hole 1404 includes a first visualization flange 1405 that extends substantially perpendicular relative to a plane of the cover plate 1222. This first visualization flange 1405 may then be laser welded or laser brazed to the interior surface of a corresponding ferrule enclosure 1220. Likewise, the second visualization hole 1406 may include a second visualization flange 1407 that extends substantially perpendicular relative to the plane of the cover plate, and this second visualization flange 1407 may be laser welded or laser brazed to the interior surface of a corresponding ferrule enclosure 1220. This ensures a watertight and airtight seal that can undergone multiple autoclave sterilization cycles.

Additionally, the window 1202 is hermetically laser brazed to the metal cover plate 1222 to further ensure a watertight and airtight seal. Additionally, the individual fibers of the fiber optic bundles (see 1210a-1210b, 1212a-1212b) are glued and polished to prevent moisture from attaching to and wicking along the optical fibers. The individual fibers may be glued together prior to inserting the fiber optic bundles (see 1210a-1210b, 1212a-1212b) into the protective ferrule enclosures 1220. The ferrule enclosures 1220 isolate the optical fibers from the cavity within the insertion tube and provide a penetration barrier between the optical fiber bundles and the optical and electronic components disposed within the insertion tube. Thus, the moisture-permeable optical fibers are portioned off from the optical and electronic components without constructing two separate mechanical housings (i.e., one housing for the illumination fibers, and another housing for the optical and electronic components).

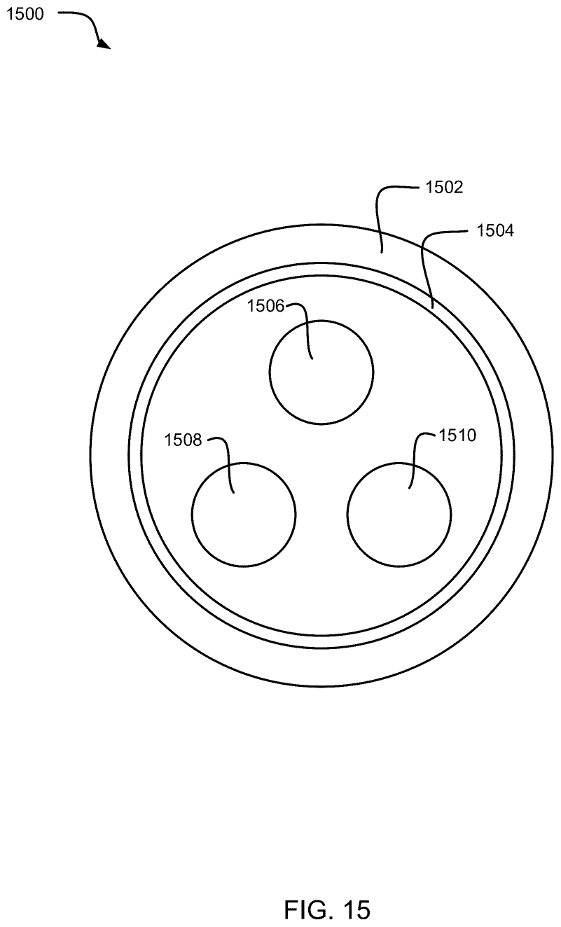

FIG. 15 is a schematic cross-sectional side view of a system 800 for cable placement within the insertion tube of an endoscope. The system 800 includes a jacket 802 which may specifically be constructed of silicone or another flexible material capable of withstanding elevated temperatures. The system 800 includes a mesh layer 804 which may be manufactured from a helix and fiber mesh. The system 800 includes one or more fiber bundles 806 and additionally includes one or more data transmission cables 808. The data transmission cables 808 may specifically include a coaxial cable.

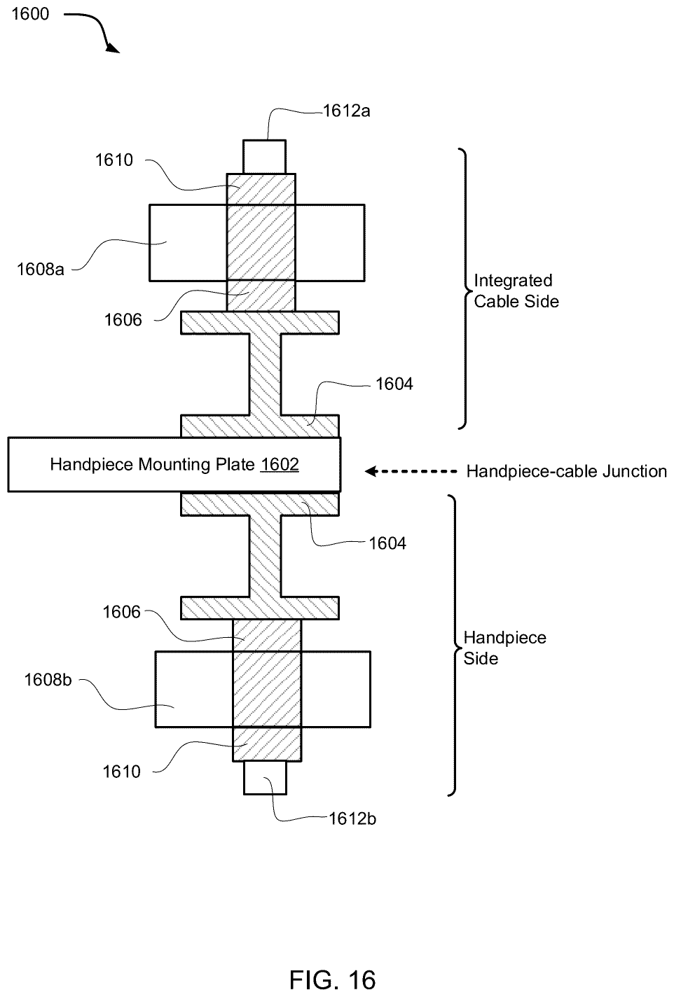

FIG. 16 is a schematic illustration of a system 1600 for preventing moisture ingress into the handpiece (see 154) at the interfaced where the integrated cable (see 502) enters the handpiece. This is referred to herein as the handpiece-cable junction. The system 1600 utilizes a glass-potted data cable connector with an embedded optical fiber bundle to form a hermetical seal to separate an interior of the handpiece from the exterior of the handpiece. This glass-to-metal connector is laser welded or laser brazed to a metal housing of the handpiece. A watertight and airtight handpiece-cable junction is formed by using glass as a sealing material to create a combined protective barrier that is resistant to pressure and temperature. This represents a significant improvement over traditional sealing materials, including O-rings and grommet seals.

The system 1600 is attached to a mounting plate 1602 of the handpiece (see 154). The mounting plate 1602 is constructed of a metal material such that components may be laser welded or laser brazed to the mounting plate 1602. The system 1600 bridges the mounting plate 1602 such that the optical/electronic components include an integrated cable side (top of FIG. 16) and a handpiece side (bottom of FIG. 16). The system 1600 enables electromagnetic illumination communication and/or data transmission communication to pass from the handpiece to the integrated cable.

The system 1600 may include glass-to-metal seals for radiofrequency (RF) conductor feedthrough at the handpiece-cable junction. The integrated cable 502 is an open system permeable to moisture during the autoclave sterilization cycle, wherein the moisture may enter the integrated cable 502 through the integrated connector (see 504). This necessitates a robust sealing method at the handpiece-cable junction to prevent moisture ingress and propagation to the handpiece electronics and other optical-electronic payload components further down the shaft of the insertion tube (see 112). The system 1600 utilizes glass-to-metal manufacturing processes to provide a hermetically sealed radiofrequency feedthrough for a video signal chain and power. Adapters may be utilized to interface between board-mounted connectors in the handpiece, to a glass potted radiofrequency feedthrough. The glass potted radiofrequency feedthrough may be mounted to a metal mounting plate 1602 that is laser welded to a rear of the handpiece. This provides a hermetic seal for the electronic signal chain.

In additional to the electronics chain, the system 1600 provides a seal for preventing the ingress of moisture into the illumination components. This may be achieved by implementing a continuous fiber optic bundle from a distal end of the insertion tube (see 112) to the integrated connector (see 504). This may further be achieved by separating the optical fiber bundles of the illumination system at the handpiece-tube junction, and again at the handpiece-cable junction. These separate fiber optic bundles then form a butt-coupled interface at the handpiece-tube junction and the handpiece-cable junction. The mounting plate 1602 for the glass potted radiofrequency feedthroughs provides a passthrough location or illumination junction location.

The system 1600 may be utilized to provide a hermetically sealed passthrough butt-coupled illumination interface at the handpiece-cable junction. A crimp tube 1604 may be formed integral to the mounting plate 1602 of the handpiece. The crimp tube 1604 is crimped on either side of the mounting plate 1602 as shown in FIG. 16. The crimp tube 1604 is crimped onto a jacketed illumination bundle 1608a on an integrated cable side and is further crimped onto a jacked illumination bundle 1608b on a handpiece side. The joints between the crimp tube 1604 and the jacketed illumination bundles 1608a, 1608b are potted with adhesive and covered by a polymer moisture barrier sleeve 1606. This prevents moisture ingress and propagation from the integrated cable side to the handpiece side. Additionally, on each of the integrated cable side and the handpiece side, a metal illumination ferrule 1612a, 1612b is laser welded 1610 into the mounting plate 1602. The corresponding optical fibers that are in electromagnetic communication with the metal illumination ferrules 1612a, 1612b are glued and polished to prevent moisture from wicking on to the optical fibers. Thus, the metal illumination ferrule 1612a at the integrated cable side is mechanically coupled to the metal illumination ferrule 1612b on the handpiece side at the mounting plate 1602 of the handpiece. This mechanical coupling prevents moisture from propagating from the integrated cable to the handpiece. This is due to the fiber optic bundles being mechanically coupled face-to-face, rather than a continuous bundle running an entire length of the endoscope assembly.

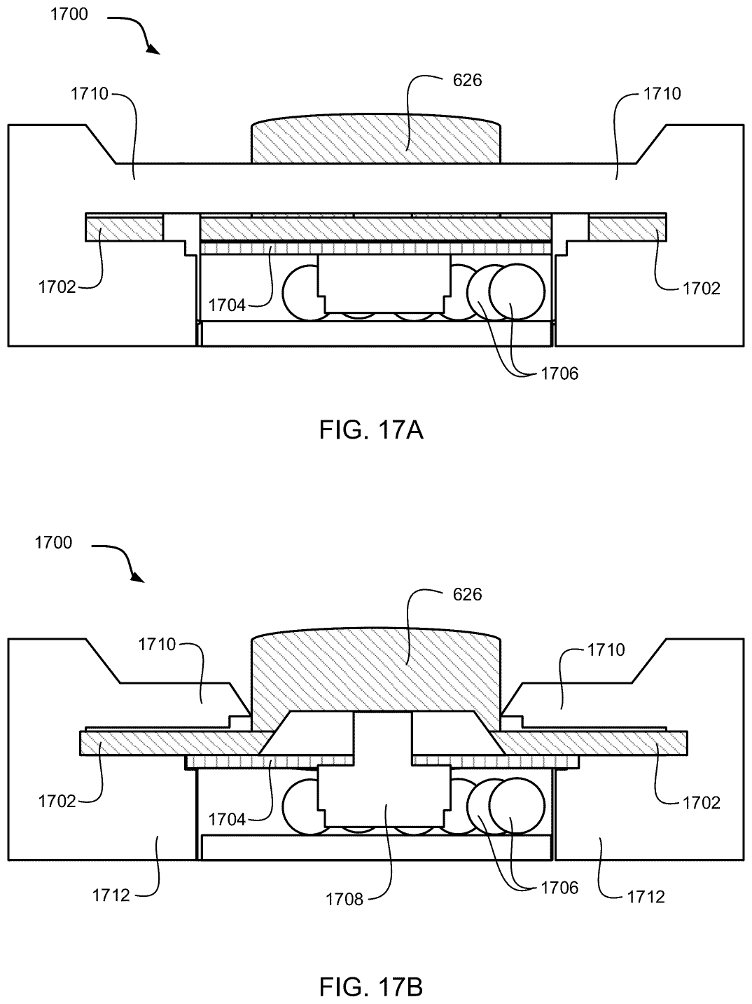

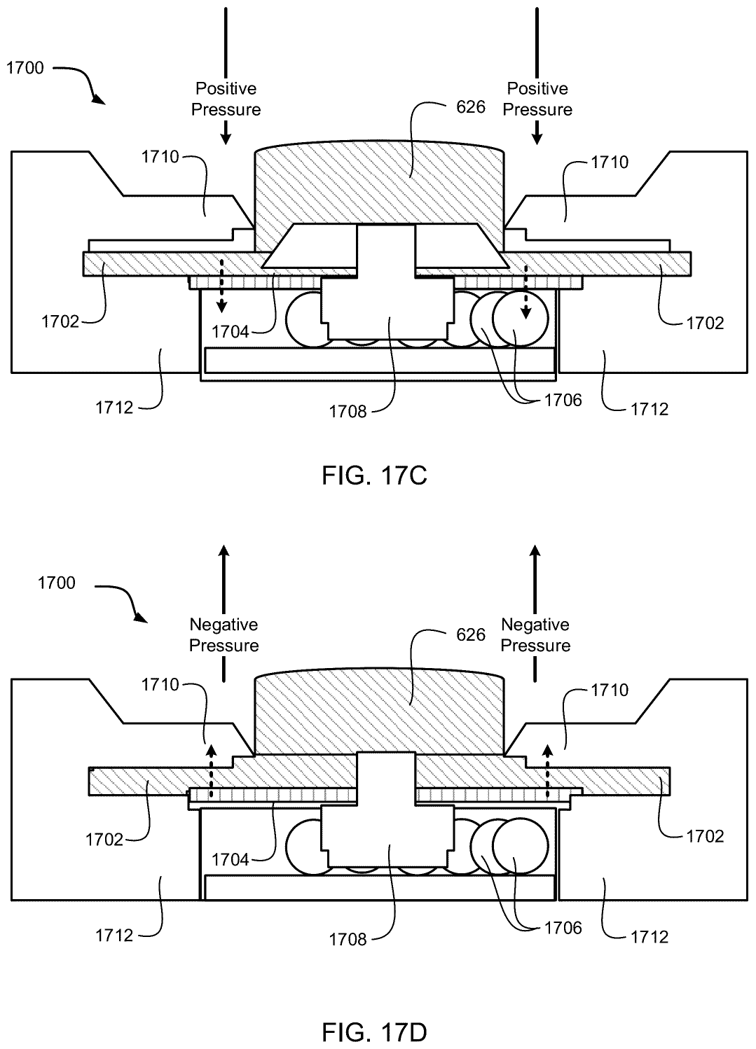

FIGS. 17A-17D illustrate a system 1700 for preventing moisture ingress through actuators on an endoscope handpiece. FIG. 17A is a schematic straight-on side view of the system 1700 in a neutral pressure position. FIG. 17B is a schematic cross-sectional side view of the system in a neutral pressure position. FIG. 17C is a schematic cross-sectional side view of the system when a seal of the system experiences a positive pressure. FIG. 17D is a schematic cross-sectional side view of the system when the seal of the system experiences a negative pressure.

The system 1700 includes an actuator 626 of the handpiece (see 154). The handpiece comprises a housing, and at least a portion of the actuator 626 is disposed through a hole formed in the housing of the handpiece. The system 1700 ensures sufficient airtight and watertight moisture sealing around the actuator 626 when the system 1700 undergoes an autoclave sterilization cycle.

The system 1700 includes an elastomer seal 1702 mounted between an upper rigid mounting body 1710 and a lower rigid mounting body 1712. The upper rigid mounting body 1710 may include a portion of the housing of the handpiece. The lower rigid mounting body 1712 may additionally include a portion of the housing of the handpiece. In such an implementation, the housing of the handpiece may include a ridge or channel cut into the thickness that accommodates the elastomer seal 1702 to be disposed therein.

The elastomer seal 1702 lays over a rigid disk 1704 that allows for a toggle mechanism 1708 of the actuator 626 to pass under a main shape of the elastomer seal 1702. The elastomer seal 1702 is disposed in between the actuator 626 and the rigid disk 1704. The rigid disk 1704 and the elastomer seal 1702 are permitted to move in the “vertical” direction such that the rigid disk 1704 and elastomer seal 1702 may move outward toward the outer housing of the handpiece and may further move inward toward a center of the housing of the handpiece. This is particularly interested in FIGS. 17C and 17D, wherein the elastomer seal 1702 and rigid disk 1704 move inward in response to positive pressure being applied to the handpiece in FIG. 17C, and wherein the elastomer seal 1702 and rigid disk 1704 move outward in response to a negative pressure being applied to the handpiece in FIG. 17D. The outward-most position of the elastomer seal 1702 and the rigid disk 1704 is constrained by the upper rigid mounting body 1710. The inward-most position of the elastomer seal 1702 and the rigid disk 1704 is constrained by a ball bearing 1706. The rigid disk 1704 is confined radially by the toggle mechanism 1708 of the actuator 626.

As shown in FIG. 17C, the system 1700 may experience a positive pressure pushing down on the actuator 626. This may specifically occur during an autoclave sterilization cycle. When the system 1700 experiences a positive pressure that exceeds a positive pressure threshold, as shown in FIG. 17C, the elastomer seal 1702 and rigid disk 1704 depress inward toward a center of the housing of the handpiece. The elastomer seal 1702 and rigid disk 1704 may depress inward until the rigid disk 1704 presses against the ball bearing 1706. The elastomer seal 1702 creates an airtight and watertight seal against the lower rigid mounting body 1712, and this seal prevents moisture from entering the internal space defined by the housing of the handpiece.

As shown in FIG. 17D, the system 1700 may experience a negative pressure pulling on the actuator 626. This may specifically occur during a vacuum phase or a dry phase of the autoclave sterilization cycle. When the system 1700 experiences the negative pressure that exceeds a negative pressure threshold, as shown in FIG. 17D, the elastomer seal 1702 is pulled outward toward the upper rigid mounting body 1710. In some cases, and as shown in FIG. 17D, the rigid disk 1704 may also be pulled outward toward the upper rigid mounting body 1710, but this is not required and may not always occur. The elastomer seal 1702 creates an airtight and watertight seal against the upper rigid mounting body 1710, and this seal prevents moisture from entering the internal space defined by the housing of the handpiece.

### EXAMPLES

The following examples pertain to preferred features of further embodiments:

Example 1 is an endoscope. The endoscope includes a handpiece and an insertion tube attached to the handpiece, wherein the insertion tube comprises a sidewall, and wherein the sidewall comprises an interior surface and an exterior surface. The endoscope includes a cover plate attached to the interior surface of the sidewall of the insertion tube, wherein the cover plate comprises an illumination hole. The endoscope includes a window attached to the cover plate. The endoscope includes a fiber optic ferrule disposed within the insertion tube, wherein the fiber optic ferrule is configured to receive a distal fiber optic bundle, wherein the fiber optic ferrule is attached to the cover plate at the illumination hole such that electromagnetic radiation transmitted by the distal fiber optic bundle passes through the illumination hole of the cover plate.

Example 2 is an endoscope as in Example 1, further comprising an optical-electronic payload disposed within an interior space defined by the sidewall of the insertion tube, and wherein at least a portion of the optical-electronic payload is disposed at a distal end of the insertion tube.

Example 3 is an endoscope as in any of Examples 1-2, wherein the cover plate further comprises an optical hole, and wherein the optical-electronic payload comprises: a lens; a prism, wherein the prism is located adjacent to the lens; and an image sensor, wherein at least one of the lens or the prism focuses electromagnetic radiation on to a pixel array of the image sensor.

Example 4 is an endoscope as in any of Examples 1-3, wherein the lens is disposed substantially adjacent to the window and is substantially aligned with the optical hole of the cover plate such that reflected electromagnetic radiation passes through the window and then passes through the lens prior to irradiating the pixel array of the image sensor.

Example 5 is an endoscope as in any of Examples 1-4, wherein the optical-electronic payload comprises: a first lens; a first prism, wherein the first prism is located adjacent to the first lens; a first image sensor, wherein at least one of the first lens or the first prism focuses electromagnetic radiation on to a first pixel array of the first image sensor; a second lens; a second prism, wherein the second prism is located adjacent to the second lens; and a second image sensor, wherein at least one of the second lens or the second prism focuses the electromagnetic radiation on to a second pixel array of the second image sensor.

Example 6 is an endoscope as in any of Examples 1-5, wherein the cover plate further comprises a first optical hole and a second optical hole; wherein the first lens is disposed substantially adjacent to the window and is substantially aligned with the first optical hole of the cover plate such that reflected electromagnetic radiation passes through the window, the first lens, and the first prism prior to irradiating the first pixel array of the first image sensor; and wherein the second lens is disposed substantially adjacent to the window and is substantially aligned with the second optical hole of the cover plate such that the reflected electromagnetic radiation passes through the window, the second lens, and the second prism prior to irradiating the second pixel array of the second image sensor.

Example 7 is an endoscope as in any of Examples 1-6, wherein the optical-electronic payload comprises a first image sensor comprising a first pixel array; wherein the optical-electronic payload further comprises a second image sensor comprising a second pixel array; wherein a plane of the first pixel array is oriented substantially parallel to a longitudinal axis of the insertion tube; and wherein a plane of the second pixel array is oriented substantially parallel to the longitudinal axis of the insertion tube.

Example 8 is an endoscope as in any of Examples 1-7, wherein the cover plate is constructed of a metal material, and wherein the cover plate is welded to the interior surface of the sidewall of the insertion tube.

Example 9 is an endoscope as in any of Examples 1-8, wherein the cover plate further comprises a metal flange located at the illumination hole, and wherein the metal flange is welded to an interior surface of the fiber optic ferrule.

Example 10 is an endoscope as in any of Examples 1-9, wherein the window is hermetically laser brazed to the cover plate.

Example 11 is an endoscope as in any of Examples 1-10, wherein the window is constructed of an optical-grade transparent material, and wherein the optical-grade transparent material comprises one or more of sapphire glass, fused silica glass, or quartz.

Example 12 is an endoscope as in any of Examples 1-11, wherein each of the fiber optic ferrule and the cover plate is constructed of a metal material, and wherein the fiber optic ferrule is welded to the cover plate.

Example 13 is an endoscope as in any of Examples 1-12, wherein the fiber optic ferrule comprises: a first fiber optic ferrule configured to receive a first distal fiber optic bundle, wherein the first distal fiber optic bundle is dedicated to transmitting a first type of electromagnetic radiation; and a second fiber optic ferrule configured to receive a second distal fiber optic bundle, wherein the second distal fiber optic bundle is dedicated to transmitting a second type of electromagnetic radiation.

Example 14 is an endoscope as in any of Examples 1-13, wherein the first type of electromagnetic radiation comprises broadband visible electromagnetic radiation; wherein the second type of electromagnetic radiation comprises one or more narrowband wavebands of electromagnetic radiation; wherein the one or more narrowband wavebands of electromagnetic radiation comprises a waveband that is 40 nanometers wide or less; and wherein the one or more narrowband wavebands is selected for advanced visualization of a scene comprising one or more of fluorescence visualization, multispectral visualization, or hyperspectral visualization.

Example 15 is an endoscope as in any of Examples 1-14, wherein the handpiece comprises a handpiece distal end and a handpiece proximal end; wherein the handpiece is attached to the insertion tube at the handpiece distal end; wherein the handpiece is attached to an integrated cable at the handpiece proximal end; wherein the integrated cable comprises a proximal fiber optic bundle.

Example 16 is an endoscope as in any of Examples 1-15, wherein the proximal fiber optic bundle forms a butt-coupled interface to the distal fiber optic bundle wherein the handpiece is attached to the integrated cable at the handpiece proximal end.

Example 17 is an endoscope as in any of Examples 1-16, wherein the distal fiber optic bundle comprises a plurality of optical fibers, and wherein at least a portion of a total length of the plurality of optical fibers is glued together to prevent moisture from wicking on to any of the plurality of optical fibers when the endoscope undergoes an autoclave sterilization cycle.

Example 18 is an endoscope as in any of Examples 1-17, further comprising: a first image sensor disposed within the insertion tube; and a second image sensor disposed within the insertion tube; wherein the fiber optic ferrule comprises a first fiber optic ferrule encasing a first distal fiber optic bundle; and wherein the fiber optic ferrule further comprises a second fiber optic ferrule encasing a second distal fiber optic bundle.

Example 19 is an endoscope as in any of Examples 1-18, further comprising a printed circuit board disposed within the insertion tube.

Example 20 is an endoscope as in any of Examples 1-19, further comprising: a first image sensor disposed within the insertion tube; a second image sensor disposed within the insertion tube; a thermal heat sink disposed within the insertion tube; and a data transmission cable disposed within the insertion tube, wherein the data transmission cable provides bidirectional electronic communication between a microcontroller disposed in the handpiece and one or more of the first image sensor or the second image sensor.

Example 21 is an endoscope. The endoscope includes an insertion tube; a handpiece attached to the insertion tube, wherein the handpiece comprises a handpiece housing, and wherein the handpiece housing comprises a mounting plate; a cable comprising a proximal end and a distal end, wherein the distal end of the cable is attached to the mounting plate of the handpiece housing to form a hermetic junction; and a connector coupled to the proximal end of the cable, wherein the connector comprises at least one opening that is permeable to moisture during an autoclave sterilization cycle; wherein the hermetic junction comprises a laser brazed seal.

Example 22 is an endoscope as in Example 21, wherein the cable comprises a fiber optic bundle and a data transmission cable.

Example 23 is an endoscope as in any of Examples 21-22, wherein the hermetic junction prevents the moisture permeated into the connector from entering an interior of the handpiece.

Example 24 is an endoscope as in any of Examples 21-23, wherein the cable comprises a fiber optic bundle; wherein the handpiece further comprises a crimp tube attached to the mounting plate; wherein the crimp tube is laser brazed or welded to the mounting plate; and wherein the crimp tube is crimped onto the fiber optic bundle of the cable to form an illumination junction.

Example 25 is an endoscope as in any of Examples 21-24, wherein a distal end of the fiber optic bundle terminates at the illumination junction; wherein a proximal end of the fiber optic bundle terminates at the connector; and wherein the connector is configured to interface with an emitter such that electromagnetic radiation pulsed by the emitter is transmitted by the fiber optic bundle to the illumination junction.

Example 26 is an endoscope as in any of Examples 21-25, wherein the illumination junction formed at the mounting plate of the handpiece is airtight and water-tight such that the moisture permeated through the connector during the autoclave sterilization cycle is prevented from entering the handpiece.

Example 27 is an endoscope as in any of Examples 21-26, further comprising: an adhesive disposed at the illumination junction; and a polymer moisture barrier sleeve disposed around the fiber optic bundle and further disposed around at least a portion of the illumination junction.

Example 28 is an endoscope as in any of Examples 21-27, wherein the hermetic junction comprises the illumination junction, and wherein the illumination junction comprises a laser welded junction comprising a glass sealing material.

Example 29 is an endoscope as in any of Examples 21-28, wherein the cable comprises a fiber optic bundle and a data transmission cable; wherein the cable is laser brazed to the mounting plate of the handpiece at the hermetic junction; and wherein the hermetic junction is sealed such that moisture permeating into the cable through the connector during the autoclave sterilization cycle is prevented from entering an interior of the handpiece.

Example 30 is an endoscope as in any of Examples 21-29, wherein the cable is laser brazed to the mounting plate of the handpiece with a glass potting material to form a glass-to-metal seal between the cable and the mounting plate.

Example 31 is an endoscope as in any of Examples 21-30, wherein the cable comprises: a fiber optic bundle; a data transmission cable; a Y-splitter attached to each of the fiber optic bundle and the data transmission cable; and a microvalve disposed within a housing of the Y-splitter; wherein the microvalve opens to release inner pressure within the cable.

Example 32 is an endoscope as in any of Examples 21-31, wherein the cable further comprises a microvalve, and wherein the microvalve opens to release inner pressure within the cable.

Example 33 is an endoscope as in any of Examples 21-32, wherein the microvalve opens automatically in response to the inner pressure within the cable exceeding an exterior pressure outside the cable.

Example 34 is an endoscope as in any of Examples 21-33, wherein the microvalve opens automatically when the endoscope undergoes a vacuum phase of the autoclave sterilization cycle.

Example 35 is an endoscope as in any of Examples 21-34, wherein the microvalve opens automatically when the endoscope undergoes a dry phase of the autoclave sterilization cycle.

Example 36 is an endoscope as in any of Examples 21-35, wherein the microvalve is in a closed position when the inner pressure within the cable does not exceed an exterior pressure outside the cable; and wherein the microvalve prevents moisture from entering the cable when the microvalve is in the closed position.

Example 37 is an endoscope as in any of Examples 21-36, wherein the connector comprises: a fiber optic ferrule configured to interface with a corresponding waveguide contact, wherein the corresponding waveguide contact is in electromagnetic communication with an emitter that pulses electromagnetic radiation; and a data connector configured to interface with a corresponding data port of a controller.

Example 38 is an endoscope as in any of Examples 21-37, wherein the emitter pulses electromagnetic radiation to illuminate a scene for endoscopic visualization; wherein the emitter pulses broadband visible electromagnetic radiation; wherein the emitter pulses narrowband electromagnetic radiation; and wherein the narrowband electromagnetic radiation is selected for fluorescence or multispectral visualization of the scene.

Example 39 is an endoscope as in any of Examples 21-38, further comprising an optical-electronic payload disposed within the insertion tube, wherein the optical-electronic payload comprises: a first image sensor; and a second image sensor; and wherein the optical-electronic payload is disposed at a distal end of the insertion tube.

Example 40 is an endoscope as in any of Examples 21-39, wherein the endoscope undergoes the autoclave sterilization cycle, and wherein the hermetic junction is sufficiently watertight to prevent moisture from reaching the optical-electronic payload during the autoclave sterilization cycle.

Example 41 is an electronic device suitable for autoclave sterilization. The electronic device includes a housing, wherein one or more electronic components is disposed within an interior space defined by the housing. The electronic device includes an actuator, wherein at least a portion of the actuator is disposed through a hole formed in the housing. The electronic device includes an elastomer seal disposed within the interior space defined by the housing. The electronic device includes a rigid disk located adjacent to the elastomer seal. The electronic device includes a ball bearing. The electronic device is such that the elastomer seal deforms outward toward an exterior of the housing in response to an internal pressure within the housing exceeding an external pressure outside the housing.

Example 42 is an electronic device as in Example 41, wherein the housing is a handpiece of an endoscope assembly, and wherein an insertion tube of the endoscope assembly is attached to the housing.

Example 43 is an electronic device as in any of Examples 41-42, wherein the actuator comprises one or more of a button or a toggle.

Example 44 is an electronic device as in any of Examples 41-43, wherein the elastomer seal deforms outward toward the exterior of the housing in response to the electronic device experiencing a negative pressure when undergoing an autoclave sterilization cycle; and wherein the elastomer seal forms a seal against the housing such that moisture is prevented from entering the interior space defined by the housing when the internal pressure exceeds the external pressure.

Example 45 is an electronic device as in any of Examples 41-44, wherein the elastomer seal is located immediately adjacent to an interior surface of the housing.

Example 46 is an electronic device as in any of Examples 41-45, wherein the elastomer seal is disposed in between a first rigid body and a second rigid body; wherein the first rigid body is the housing of the electronic device; and wherein the second rigid body is the rigid disk.

Example 47 is an electronic device as in any of Examples 41-46, wherein the elastomer seal comprises a substantially cylindrical geometry; wherein the rigid disk comprises a substantially cylindrical geometry; and wherein a diameter of the elastomer seal is greater than a diameter of the rigid disk.

Example 48 is an electronic device as in any of Examples 41-47, wherein the ball bearing comprises a plurality of balls movable on a substantially cylindrical track; and wherein a diameter of the substantially cylindrical track is smaller than the diameter of the elastomer seal or the diameter of the rigid disk.

Example 49 is an electronic device as in any of Examples 41-48, wherein a position of the rigid disk is movable outward toward the housing and inward away from the housing; wherein an outward-most position of the rigid disk is constrained by the housing; and wherein an inward-most position of the rigid disk is constrained by the ball bearing.

Example 50 is an electronic device as in any of Examples 41-49, wherein the elastomer seal and the rigid disk depress inward toward the ball bearing in response to the electronic device experiencing a positive pressure that exceeds a positive pressure threshold.

Example 51 is an electronic device as in any of Examples 41-50, wherein the positive pressure on the electronic device exceeds the positive pressure threshold when the electronic device undergoes an autoclave sterilization cycle; and wherein the elastomer seal prevents moisture from entering the interior space defined by the housing when the elastomer seal and the rigid disk depress inward toward the ball bearing.

Example 52 is an electronic device as in any of Examples 41-51, wherein a radial position of the rigid disk is confined by a toggle mechanism of the actuator.

Example 53 is an electronic device as in any of Examples 41-52, wherein the housing is a handpiece of an endoscope assembly; wherein an insertion tube of the endoscope assembly is attached to a distal end of the housing to form a handpiece-tube junction; wherein a cable of the endoscope assembly is attached to a proximal end of the housing to form a handpiece-cable junction; and wherein each of the handpiece-tube junction and the handpiece-cable junction is hermetically sealed.

Example 54 is an electronic device as in any of Examples 41-53, wherein the cable terminates at a connector that permits moisture to enter the cable, and wherein the handpiece-cable junction is hermetically sealed such that moisture is prevented from passing from the cable into the interior space defined by the housing.

Example 55 is an electronic device as in any of Examples 41-54, wherein the cable comprises a data transmission cable and a fiber optic bundle.

Example 56 is an electronic device as in any of Examples 41-55, wherein the endoscope assembly comprises two or more image sensors disposed within the insertion tube, and wherein the two or more image sensors are located at a distal end of the insertion tube.

Example 57 is an electronic device as in any of Examples 41-56, wherein the endoscope assembly comprises at least the housing, the insertion tube, the cable, and the two or more image sensors; and wherein the endoscope assembly is sealed such that the endoscope assembly undergoes an autoclave sterilization cycle without permitting moisture to enter an interior space of the insertion tube or the interior space defined by the housing.

Example 58 is an electronic device as in any of Examples 41-57, wherein the cable comprises a proximal optical fiber bundle that receives electromagnetic radiation pulsed by an emitter; wherein the proximal optical fiber bundle forms a butt-coupled interface with a distal optical fiber bundle at the handpiece-cable junction; and wherein the distal optical fiber bundle transmits the electromagnetic radiation pulsed by the emitter to a distal end of the insertion tube.

Example 59 is an electronic device as in any of Examples 41-58, wherein the electromagnetic radiation pulsed by the emitter comprises broadband visible electromagnetic radiation and further comprises a narrowband near-infrared waveband of electromagnetic radiation that is selected to excite a fluorescent reagent or fluorescent tissue.

Example 60 is an electronic device as in any of Examples 41-59, wherein the electromagnetic radiation pulsed by the emitter comprises broadband visible electromagnetic radiation and further comprises a narrowband multispectral waveband of electromagnetic radiation that is selected to elicit a spectral response from a tissue or penetrate through a tissue.

Example 61 is an electronic device as in any of Examples 41-60, wherein the electronic device further includes any of the systems, methods, devices, components, or functionalities described in connection with any of Examples 1-40.

Example 62 is an endoscope as in any of Examples 1-20, wherein the endoscope further includes any of the systems, methods, devices, components, or functionalities described in connection with any of Examples 21-60.

Example 63 is an endoscope as in any of Examples 21-40, wherein the endoscope further includes any of the systems, methods, devices, components, or functionalities described in connection with any of Examples 1-20 and/or Examples 41-60.

It will be appreciated that various features disclosed herein provide significant advantages and advancements in the art. The following claims are exemplary of some of those features.

In the foregoing Detailed Description of the Disclosure, various features of the disclosure are grouped together in a single embodiment for the purpose of streamlining the disclosure. This method of disclosure is not to be interpreted as reflecting an intention that the claimed disclosure requires more features than are expressly recited in each claim. Rather, inventive aspects lie in less than all features of a single foregoing disclosed embodiment.

It is to be understood that any features of the above-described arrangements, examples, and embodiments may be combined in a single embodiment comprising a combination of features taken from any of the disclosed arrangements, examples, and embodiments.

It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and arrangements.

Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

Further, where appropriate, functions described herein can be performed in one or more of: hardware, software, firmware, digital components, or analog components. For example, one or more application specific integrated circuits (ASICs) or field programmable gate arrays (FPGAs) can be programmed to carry out one or more of the systems and procedures described herein. Certain terms are used throughout the following description and claims to refer to particular system components. As one skilled in the art will appreciate, components may be referred to by different names. This document does not intend to distinguish between components that differ in name, but not function.

The foregoing description has been presented for the purposes of illustration and description. It is not intended to be exhaustive or to limit the disclosure to the precise form disclosed. Many modifications and variations are possible in light of the above teaching. Further, it should be noted that any or all the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. An endoscope comprising:
a handpiece;
an insertion tube attached to the handpiece, wherein the insertion tube comprises a sidewall, and wherein the sidewall comprises an interior surface and an exterior surface;
a cover plate attached to the interior surface of the sidewall of the insertion tube, wherein the cover plate comprises an illumination hole;
a window attached to the cover plate; and
a fiber optic ferrule disposed within the insertion tube, wherein the fiber optic ferrule is configured to receive a distal fiber optic bundle;
wherein the fiber optic ferrule is attached to the cover plate at the illumination hole such that electromagnetic radiation transmitted by the distal fiber optic bundle passes through the illumination hole of the cover plate.

2. The endoscope of claim 1, further comprising an optical-electronic payload disposed within an interior space defined by the sidewall of the insertion tube;
wherein at least a portion of the optical-electronic payload is disposed at a distal end of the insertion tube;
wherein the cover plate further comprises an optical hole; and
wherein the optical-electronic payload comprises:
a lens;
a prism, wherein the prism is located adjacent to the lens; and
an image sensor, wherein at least one of the lens or the prism focuses electromagnetic radiation on to a pixel array of the image sensor.

3. The endoscope of claim 2, wherein the lens is disposed substantially adjacent to the window and is substantially aligned with the optical hole of the cover plate such that reflected electromagnetic radiation passes through the window and then passes through the lens prior to irradiating the pixel array of the image sensor.

4. The endoscope of claim 2, wherein the optical-electronic payload comprises:
a first lens;
a first prism, wherein the first prism is located adjacent to the first lens;
a first image sensor, wherein at least one of the first lens or the first prism focuses electromagnetic radiation on to a first pixel array of the first image sensor;
a second lens;
a second prism, wherein the second prism is located adjacent to the second lens; and
a second image sensor, wherein at least one of the second lens or the second prism focuses the electromagnetic radiation on to a second pixel array of the second image sensor.

5. The endoscope of claim 4, wherein the cover plate further comprises a first optical hole and a second optical hole;
wherein the first lens is disposed substantially adjacent to the window and is substantially aligned with the first optical hole of the cover plate such that reflected electromagnetic radiation passes through the window, the first lens, and the first prism prior to irradiating the first pixel array of the first image sensor; and
wherein the second lens is disposed substantially adjacent to the window and is substantially aligned with the second optical hole of the cover plate such that the reflected electromagnetic radiation passes through the window, the second lens, and the second prism prior to irradiating the second pixel array of the second image sensor.

6. The endoscope of claim 1, wherein the cover plate is constructed of a metal material, and wherein the cover plate is welded to the interior surface of the sidewall of the insertion tube.

7. The endoscope of claim 1, wherein the cover plate further comprises a metal flange located at the illumination hole, and wherein the metal flange is welded to an interior surface of the fiber optic ferrule.

8. The endoscope of claim 1, wherein the window is hermetically laser brazed to the cover plate.

9. An endoscope comprising:
an insertion tube;
a handpiece attached to the insertion tube, wherein the handpiece comprises a handpiece housing, and wherein the handpiece housing comprises a mounting plate;
a cable comprising a proximal end and a distal end, wherein the distal end of the cable is attached to the mounting plate of the handpiece housing to form a hermetic junction; and
a connector coupled to the proximal end of the cable, wherein the connector comprises at least one opening that is permeable to moisture during an autoclave sterilization cycle;
wherein the hermetic junction comprises a laser brazed seal.

10. The endoscope of claim 9, wherein the cable comprises a fiber optic bundle and a data transmission cable; and
wherein the hermetic junction prevents the moisture permeated into the connector from entering an interior of the handpiece.

11. The endoscope of claim 9, wherein the cable comprises a fiber optic bundle;
wherein the handpiece further comprises a crimp tube attached to the mounting plate;
wherein the crimp tube is laser brazed or welded to the mounting plate; and
wherein the crimp tube is crimped onto the fiber optic bundle of the cable to form an illumination junction.

12. The endoscope of claim 11, wherein the illumination junction formed at the mounting plate of the handpiece is airtight and water-tight such that the moisture permeated through the connector during the autoclave sterilization cycle is prevented from entering the handpiece; and
wherein the endoscope further comprises:
an adhesive disposed at the illumination junction; and
a polymer moisture barrier sleeve disposed around the fiber optic bundle and further disposed around at least a portion of the illumination junction.

13. The endoscope of claim 9, wherein the cable comprises a fiber optic bundle and a data transmission cable;
wherein the cable is laser brazed to the mounting plate of the handpiece at the hermetic junction;
wherein the hermetic junction is sealed such that moisture permeating into the cable through the connector during the autoclave sterilization cycle is prevented from entering an interior of the handpiece; and
wherein the cable is laser brazed to the mounting plate of the handpiece with a glass potting material to form a glass-to-metal seal between the cable and the mounting plate.

14. The endoscope of claim 9, wherein the cable comprises:
a fiber optic bundle;
a data transmission cable;
a Y-splitter attached to each of the fiber optic bundle and the data transmission cable; and
a microvalve disposed within a housing of the Y-splitter;
wherein the microvalve opens automatically in response to the inner pressure within the cable exceeding an exterior pressure outside the cable;
wherein the microvalve is in a closed position when the inner pressure within the cable does not exceed an exterior pressure outside the cable; and
wherein the microvalve prevents moisture from entering the cable when the microvalve is in the closed position.

15. An electronic device suitable for autoclave sterilization, wherein the electronic device comprises:
a housing, wherein one or more electronic components is disposed within an interior space defined by the housing;
an actuator, wherein at least a portion of the actuator is disposed through a hole formed in the housing;
an elastomer seal disposed within the interior space defined by the housing;
a rigid disk located adjacent to the elastomer seal; and
a ball bearing;
wherein the elastomer seal deforms outward toward an exterior of the housing in response to an internal pressure within the housing exceeding an external pressure outside the housing.

16. The electronic device of claim 15, wherein the housing is a handpiece of an endoscope assembly, and wherein an insertion tube of the endoscope assembly is attached to the housing; and
wherein the actuator comprises one or more of a button or a toggle.

17. The electronic device of claim 15, wherein the elastomer seal deforms outward toward the exterior of the housing in response to the electronic device experiencing a negative pressure when undergoing an autoclave sterilization cycle; and
wherein the elastomer seal forms a seal against the housing such that moisture is prevented from entering the interior space defined by the housing when the internal pressure exceeds the external pressure.

18. The electronic device of claim 15, wherein the elastomer seal is disposed in between a first rigid body and a second rigid body;
wherein the first rigid body is the housing of the electronic device; and
wherein the second rigid body is the rigid disk.

19. The electronic device of claim 15, wherein the elastomer seal comprises a substantially cylindrical geometry;
wherein the rigid disk comprises a substantially cylindrical geometry;
wherein a diameter of the elastomer seal is greater than a diameter of the rigid disk;
wherein the ball bearing comprises a plurality of balls movable on a substantially cylindrical track; and
wherein a diameter of the substantially cylindrical track is smaller than the diameter of the elastomer seal or the diameter of the rigid disk.

20. The electronic device of claim 15, wherein a position of the rigid disk is movable outward toward the housing and inward away from the housing;
wherein an outward-most position of the rigid disk is constrained by the housing;
wherein an inward-most position of the rigid disk is constrained by the ball bearing; and
wherein the elastomer seal and the rigid disk depress inward toward the ball bearing in response to the electronic device experiencing a positive pressure that exceeds a positive pressure threshold.
